SEQUENCE LISTING (38 sequences)

SEQ ID NO: 1 - DNA, 1887 bp, synthetic construct
  source 1..1887: mol_type = other DNA
atgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtccggtggcggtggctccggcggtggtgggtcgggtggcggcggatctcaggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaagctggaggatcccaaagtgcccagggattgtggttgtaagccttgcatatgtacagtcccagaagtattctgggctctggtggtggtggccggcgtgctgttttgttacggcctgctcgtgacagtggccctgtgcgtgatctggaccaacagcagaagaaacagaggcggccagtccgactacatgaacatgaccccccgcagacccggcctgaccagaaagccttaccagccttacgcccctgccagagatttcgccgcctacagacccagagccaagttcagcagaagcgccgagacagccgccaacctgcaggaccctaaccagctgttcaacgagctgaacctgggcagacgggaagagttcgacgtgctggaaaagaagagagccagggaccccgagatgggcggcaagcagcagagaagaagaaacccccaggaaggcgtgtacaacgccctgcagaaagacaagatggccgaggcctacagcgagatcggcacaaagggcgagagaaggcggggcaagggacacgatggcctgttccagggcctgagcaccgccaccaaggacacattcgatgccctgcacatgcagacactggcccccagatga

SEQ ID NO: 2 - protein, 628 aa, synthetic construct
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTSGGGGSGGGGSGGGGSQVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIKLEDPKVPRDCGCKPCICTVPEVFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 3 - DNA, 486 bp, synthetic construct
  source 1..486: mol_type = other DNA
atgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcc

SEQ ID NO: 4 - protein, 162 aa, synthetic construct
  SIGNAL 1..29
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 5 - DNA, 45 bp, synthetic construct
  source 1..45: mol_type = other DNA
ggtggcggtggctccggcggtggtgggtcgggtggcggcggatct

SEQ ID NO: 6 - DNA, 750 bp, synthetic construct
  source 1..750: mol_type = other DNA
caggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaagctggag

SEQ ID NO: 7 - protein, 250 aa, synthetic construct
QVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIKLE

SEQ ID NO: 8 - DNA, 1968 bp, synthetic construct
  source 1..1968: mol_type = other DNA
atgggcgtgcccacccagctgctgggactgctgctgctgtggatcaccgacgccatctgccaggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaagctggaggatcccaaagtgcccagggattgtggttgtaagccttgcatatgtacagtcccagaagtattctgggctctggtggtggtggccggcgtgctgttttgttacggcctgctcgtgacagtggccctgtgcgtgatctggaccaacagcagaagaaacagaggcggccagtccgactacatgaacatgaccccccgcagacccggcctgaccagaaagccttaccagccttacgcccctgccagagatttcgccgcctacagacccagagccaagttcagcagaagcgccgagacagccgccaacctgcaggaccctaaccagctgttcaacgagctgaacctgggcagacgggaagagttcgacgtgctggaaaagaagagagccagggaccccgagatgggcggcaagcagcagagaagaagaaacccccaggaaggcgtgtacaacgccctgcagaaagacaagatggccgaggcctacagcgagatcggcacaaagggcgagagaaggcggggcaagggacacgatggcctgttccagggcctgagcaccgccaccaaggacacattcgatgccctgcacatgcagacactggcccccagatgcggccgcgagggcagaggaagtcttctaacatgcggtgacgtggaggagaatcccggccctaggatgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcctga

SEQ ID NO: 9 - protein, 655 aa, synthetic construct
  REGION 1..471
  REGION 472..655
  SIGNAL 494..522
MGVPTQLLGLLLLWITDAICQVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIKLEDPKVPRDCGCKPCICTVPEVFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPRCGREGRGSLLTCGDVEENPGPRMKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 10 - DNA, 20 bp, synthetic construct
  source 1..20: mol_type = other DNA
gtggccctgtggatacacac

SEQ ID NO: 11 - DNA, 21 bp, synthetic construct
  source 1..21: mol_type = other DNA
acaacagcagctattgtcccg

SEQ ID NO: 12 - DNA, 22 bp, synthetic construct
  source 1..22: mol_type = other DNA
aatcagataccgcaatgaccac

SEQ ID NO: 13 - DNA, 22 bp, synthetic construct
  source 1..22: mol_type = other DNA
cagaagttgtttgggatggtgt

SEQ ID NO: 14 - DNA, 20 bp, synthetic construct
  source 1..20: mol_type = other DNA
acatctgctggaaggtggac

SEQ ID NO: 15 - DNA, 20 bp, synthetic construct
  source 1..20: mol_type = other DNA
ttgctgacaggatgcagaag

SEQ ID NO: 16 - protein, 29 aa, synthetic construct
MKILKPYMRNTSISCYLCFLLNSHFLTEA

SEQ ID NO: 17 - protein, 201 aa, synthetic construct
  REGION 1..3: note = BamH1 site
  REGION 4..20: note = IgG hing
  REGION 21..201: note = CD28zeta
DPKVPRDCGCKPCICTVPEVFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 18 - protein, 18 aa, synthetic construct
EGRGSLLTCGDVEENPGP

SEQ ID NO: 19 - DNA, 489 bp, synthetic construct
  source 1..489: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 20 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 21 - protein, 15 aa, synthetic construct
GGGGSGGGGSGGGGS

SEQ ID NO: 22 - DNA, 735 bp, synthetic construct
  source 1..735: mol_type = other DNA
  variation 55: note = D55E
  variation 56: note = G56A
caggtgcagctggtgcagtccggagcagaggtgaagaagccaggaagctccgtgaaggtgtcttgcaaggccagcggctataccttctctaactacctgatgaattgggtgaggcaggcaccaggacagggactggagtggatgggccgcatcgatccttatgacggcgatatcgactacaaccagaacttcaaggatcgggtgaccatcacagccgacaagagcacctccacagcctatatggagctgtctagcctgcggagcgaggataccgccgtgtactattgtgccagaggctacggcacagcctatggagtggactactggggacagggcaccctggtgacagtgtcctctggtggcggtggctccggcggtggtgggtcgggtggcggcggatctgacatcgtgatgacccagtcccctgattctctggccgtgtctctgggagagagggcaacaatcaactgcagagcctctgagagcgtggacaactacggcatcagcttcatgaattggtatcagcagaagccaggccagccccctaagctgctgatctacgcagcaagcaggcagggatccggagtgcccgaccggttctccggatctggaagcggaaccgacttcaccctgacaatcagctccctgcaggccgaggatgtggccgtgtactattgtcagcagtccaaggaggtgccatggacctttggcggaggaacaaagctggagatcaag

SEQ ID NO: 23 - protein, 245 aa, synthetic construct
  VARIANT 55: note = D55E
  VARIANT 56: note = G56A
QVQLVQSGAEVKKPGSSVKVSCKASGYTFSNYLMNWVRQAPGQGLEWMGRIDPYDGDIDYNQNFKDRVTITADKSTSTAYMELSSLRSEDTAVYYCARGYGTAYGVDYWGQGTLVTVSSGGGGSGGGGSGGGGSDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIK

SEQ ID NO: 24 - protein, 200 aa, synthetic construct
  REGION 1..17: note = human IgG1 hinge
  REGION 21..88: note = CD28
  REGION 89..200: note = zeta
DLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - DNA, 600 bp, synthetic construct
  source 1..600: mol_type = other DNA
  misc_feature 1..51: note = human IgG1 hinge
  misc_feature 61..264: note = CD28
  misc_feature 265..600: note = zeta
gatctcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 26 - protein, 622 aa, synthetic construct
  REGION 1..162: note = IL15
  REGION 163..177: note = linker
  REGION 178..422: note = humanized scFv IL13Ra2 clone 47
  REGION 432..439: note = human IgG1 hinge
  REGION 442..510: note = CD28
  REGION 511..622: note = zeta
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSQVQLVQSGAEVKKPGSSVKVSCKASGYTFSNYLMNWVRQAPGQGLEWMGRIDPYDGDIDYNQNFKDRVTITADKSTSTAYMELSSLRSEDTAVYYCARGYGTAYGVDYWGQGTLVTVSSGGGGSGGGGSGGGGSDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIKDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1869 bp, synthetic construct
  source 1..1869: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggtggcggtggctccggcggtggtgggtcgggtggcggcggatctcaggtgcagctggtgcagtccggagcagaggtgaagaagccaggaagctccgtgaaggtgtcttgcaaggccagcggctataccttctctaactacctgatgaattgggtgaggcaggcaccaggacagggactggagtggatgggccgcatcgatccttatgacggcgatatcgactacaaccagaacttcaaggatcgggtgaccatcacagccgacaagagcacctccacagcctatatggagctgtctagcctgcggagcgaggataccgccgtgtactattgtgccagaggctacggcacagcctatggagtggactactggggacagggcaccctggtgacagtgtcctctggtggcggtggctccggcggtggtgggtcgggtggcggcggatctgacatcgtgatgacccagtcccctgattctctggccgtgtctctgggagagagggcaacaatcaactgcagagcctctgagagcgtggacaactacggcatcagcttcatgaattggtatcagcagaagccaggccagccccctaagctgctgatctacgcagcaagcaggcagggatccggagtgcccgaccggttctccggatctggaagcggaaccgacttcaccctgacaatcagctccctgcaggccgaggatgtggccgtgtactattgtcagcagtccaaggaggtgccatggacctttggcggaggaacaaagctggagatcaaggatctcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 28 - protein, 593 aa, synthetic construct
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSQVQLVQSGAEVKKPGSSVKVSCKASGYTFSNYLMNWVRQAPGQGLEWMGRIDPYDGDIDYNQNFKDRVTITADKSTSTAYMELSSLRSEDTAVYYCARGYGTAYGVDYWGQGTLVTVSSGGGGSGGGGSGGGGSDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIKDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 402 bp, synthetic construct
  source 1..402: mol_type = other DNA
ggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 30 - protein, 133 aa, synthetic construct
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 31 - protein, 245 aa, synthetic construct
QVQLVQSGAEVKKPGSSVKVSCKASGYTFSNYLMNWVRQAPGQGLEWMGRIDPYEGDIDYNQNFKDRVTITADKSTSTAYMELSSLRSEDTAVYYCARGYGTAYGVDYWGQGTLVTVSSGGGGSGGGGSGGGGSDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIK

SEQ ID NO: 32 - protein, 245 aa, synthetic construct
QVQLVQSGAEVKKPGSSVKVSCKASGYTFSNYLMNWVRQAPGQGLEWMGRIDPYDADIDYNQNFKDRVTITADKSTSTAYMELSSLRSEDTAVYYCARGYGTAYGVDYWGQGTLVTVSSGGGGSGGGGSGGGGSDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIK

SEQ ID NO: 33 - protein, 245 aa, synthetic construct
QVQLVQSGAEVKKPGSSVKVSCKASGYTFSNYLMNWVRQAPGQGLEWMGRIDPYDGEIDYNQNFKDRVTITADKSTSTAYMELSSLRSEDTAVYYCARGYGTAYGVDYWGQGTLVTVSSGGGGSGGGGSGGGGSDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIK

SEQ ID NO: 34 - protein, 245 aa, synthetic construct
QVQLVQSGAEVKKPGSSVKVSCKASGYTFSNYLMNWVRQAPGQGLEWMGRIDPYEADIDYNQNFKDRVTITADKSTSTAYMELSSLRSEDTAVYYCARGYGTAYGVDYWGQGTLVTVSSGGGGSGGGGSGGGGSDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIK

SEQ ID NO: 35 - protein, 245 aa, synthetic construct
QVQLVQSGAEVKKPGSSVKVSCKASGYTFSNYLMNWVRQAPGQGLEWMGRIDPYDAEIDYNQNFKDRVTITADKSTSTAYMELSSLRSEDTAVYYCARGYGTAYGVDYWGQGTLVTVSSGGGGSGGGGSGGGGSDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIK

SEQ ID NO: 36 - protein, 422 aa, synthetic construct
  REGION 1..29: note = signal peptide
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSQVQLVQSGAEVKKPGSSVKVSCKASGYTFSNYLMNWVRQAPGQGLEWMGRIDPYDGDIDYNQNFKDRVTITADKSTSTAYMELSSLRSEDTAVYYCARGYGTAYGVDYWGQGTLVTVSSGGGGSGGGGSGGGGSDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIK

SEQ ID NO: 37 - protein, 393 aa, synthetic construct
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSQVQLVQSGAEVKKPGSSVKVSCKASGYTFSNYLMNWVRQAPGQGLEWMGRIDPYDGDIDYNQNFKDRVTITADKSTSTAYMELSSLRSEDTAVYYCARGYGTAYGVDYWGQGTLVTVSSGGGGSGGGGSGGGGSDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIK

SEQ ID NO: 38 - protein, 29 aa, synthetic construct
MRISKPHLRSISIQCYLCLLLNSHFLTEA

(19) United States
(12) Patent Application Publication
(10) Pub. No.: US 2025/0319128 A1
(43) Pub. Date: Oct. 16, 2025

(54) IL15-MODIFIED CAR T CELLS FOR DUAL TARGETING

(71) Applicant: Northwestern University, Evanston, IL (US)

(72) Inventor: Irina V. Balyasnikova, Evanston, IL (US)

(21) Appl. No.: 18/866,365

(22) PCT Filed: May 16, 2023
(86) PCT No.: PCT/US2023/067085

§ 371 Date: Nov. 15, 2024

Related U.S. Application Data
(60) Provisional application No. 63/342,234, filed on May 16, 2022.
(60) Provisional application No. 63/478,352, filed on Jan. 3, 2023.

(51) Int. Cl. A61K 35/17; A61K 40/11; A61K 40/31; A61K 40/42; A61P 35/00; C07K 14/54; C07K 16/28; C12N 15/86

(52) U.S. Cl. CPC A61K 35/17; A61K 40/11; A61K 40/31; A61K 40/4217; A61K 40/4234; A61P 35/00; C07K 14/5443; C07K 16/2866; C12N 15/86; A61K 2239/21; A61K 2239/47; C07K 2317/622; C07K 2319/00; C12N 2740/10043

(57) ABSTRACT

Described herein are engineered polynucleotides comprising chimeric antigen receptors (CAR) comprising a single chain antibody to IL13Rα2 (scFv); and IL15. The encoded proteins are expressed as IL13Rα2 scFv/IL15 fusion proteins, as well as soluble IL15. Also, described herein are CAR T cells expressing the encoded proteins, and methods of their use.

### CROSS-REFERENCE TO RELATED APPLICATIONS

This application claims priority to U.S. Provisional Application No. 63/478,352 filed on Jan. 3, 2023, and U.S. Provisional Application No. 63/342,234 filed on May 16, 2022, the contents of which are incorporated by reference in their entireties.

### STATEMENT REGARDING FEDERALLY SPONSORED RESEARCH

This invention was made with government support under grant number 1R01NS 106379-01A1 awarded by the National Institutes of Health. The government has certain rights in this invention.

### REFERENCE TO AN ELECTRONIC SEQUENCE LISTING

The contents of the electronic sequence listing (702581.02325.xml; Size: 56,858 bytes; and Date of Creation: May 16, 2023) is herein incorporated by reference in its entirety.

### BACKGROUND

Glioblastoma (GBM) is the most common primary brain tumor in adults. Despite improvements in surgical and radio-chemotherapy techniques, GBM remains a devastating diagnosis as current treatment regimens provide no cure and short survival.^{1,2} The success of T cells modified with chimeric antigen receptor (CAR) in the treatment of blood cancers^{3,4} inspired the investigation of CAR T cells in solid tumors^{5, 6}, including GBM.⁷⁻⁹ However, multiple barriers exist in GBM that continue to hinder the efficacy of CAR-T cells, including (i) heterogeneity in the antigen expression or concerns of on target/off cancer toxicities^{7,8}, (ii) the immunosuppressive tumor microenvironment (TME)^{10, 11}, and (iii) inefficient trafficking to glioma sitescrossing^{12, 13} all contribute to poor performance of CAR T cells in GBM. Therefore, strategies for increasing the efficacy of CAR-T cells in GBM are needed.

### SUMMARY OF THE INVENTION

Disclosed herein are compositions and methods useful for targeting tumor cells and myeloid-derived suppressor cells in glioblastoma.

In an aspect, provided herein is an engineered polynucleotide encoding a fusion protein, the fusion protein comprising a chimeric antigen receptor comprising a single chain antibody to IL13Rα2 (scFv); and interleukin 15 (IL15). In some embodiments, the scFv and the IL15 are separated by a linker. In some embodiments, the linker is (Gly₄Ser)₃. In some embodiments, the chimeric antigen receptor further comprises a transmembrane domain, wherein the scFv is between the IL15 and the transmembrane domain. In some embodiments the sequence encoding the scFv comprises a sequence having at least 95% identity to SEQ ID NO: 23. In some embodiments, the sequence encoding the IL15 comprises a sequence having at least 95% identity to SEQ ID NO. 29. In some embodiments, the engineered polynucleotide comprises a sequence having at least 95% sequence identity to SEQ ID NO: 37. In some embodiments, the polynucleotide is operably linked to an exogenous promoter sequence capable of expressing the polynucleotide in a host cell. In some embodiments, the polynucleotide is packaged in a vector for delivery. In some embodiments, the vector is a retroviral vector.

In another aspect, provided herein is an engineered polynucleotide encoding a chimeric antigen receptor, the chimeric antigen receptor comprising a single chain antibody to IL13Rα2 (scFv); and a secretory interleukin 15 (IL15s). In some embodiments, the polynucleotide is operably linked to an exogenous promoter sequence capable of expressing the polynucleotide in a host cell. In some embodiments, the polynucleotide is packaged in a vector for delivery. In some embodiments, the vector is a retroviral vector.

Also provided herein is a fusion protein encoded by any of the engineered polynucleotides described herein.

Also provided herein is a host cell comprising any of the engineered polynucleotides, fusion proteins, or proteins described herein. In some embodiments, the host cell is a T cell.

Also provided herein is a pharmaceutical composition comprising any of the engineered polynucleotides, fusion proteins, proteins, or host cells described herein, and a pharmaceutically acceptable delivery vehicle.

Also provided herein is a method of treating a cancer in a subject, the method comprising administering to the subject a therapeutically effective amount of any of the pharmaceutical compositions described herein. In some embodiments, the cancer is glioblastoma.

Also provided herein is a method of altering a tumor microenvironment in a subject, the method comprising administering to the subject any of the pharmaceutical compositions described herein in an amount effective to alter the tumor microenvironment. In some embodiments, altering the tumor microenvironment comprises reducing the amount of myeloid-derived suppressor cells in the tumor microenvironment. In some embodiments, altering the tumor microenvironment comprises reducing the levels of at least one of IL10, arginase 1, and TGF-β. In some embodiments, altering the tumor microenvironment comprises increasing the frequency of NK cells and B cells in the tumor microenvironment.

### BRIEF DESCRIPTION OF THE DRAWINGS

FIGS. 1A-1D. Expression of IL15Rα in the immune infiltrates of tumor and peripheral blood of GBM patients. A. The analysis of the TCGA data set for the RNA expression of IL15Rα shows that GBM (n=152) expresses IL15Rα at significantly higher levels than oligodendroglioma (n=191), oligoastrocytoma (n=130), and astrocytoma (n=194) tumors (adj. P<0.001, Tukey's Honest Significant Difference test). B. Spearman Rank Correlation analysis of GBMs from TCGA (n=156) and CGGA (n=225) of RNA-seq data sets shows that IL15Rα expression correlates with expression of CD163⁺, a marker of infiltrating macrophages. C. Immunostaining of GBM patient tissue section for the expression of IL15Rα and immune cells. D. Flow cytometry analysis of IL15Rα expression on the surface of immune cells harvested from GBM microenvironment and peripheral blood of patients (paired samples, n=4). Isotype control IgG served as a negative control to anti-IL15RA. Representative histogram shows IL15Rα detection in CD45⁺ CD11b⁺ cells. The data show a higher expression of IL15Rα in many CD45⁻ cell types, including subsets of monocytes, granulocytes, and B cells harvested from tumors compared to cells purified from peripheral blood. Paired t-test. ***p<0.001.

FIGS. 2A-2E. Expression of IL15Rα in brain tumor tissue of murine models of GBM. A. The diagram shows the experimental set up used for generation of Gr1+ cells for downstream analysis of IL15Rα expression. B. RNAseq analysis of Gr1⁺ cells harvested from the brain and spleens (paired samples) of mice bearing established CT2A tumors Gr1⁺ cells were harvested from 10 mice in three independent experiments. C. RT-qPCR analysis of IL15Rα mRNA expression in Gr1⁺ cells harvested from the brains and spleens of mice bearing CT2A and GL261. D. Flow cytometry analysis of IL15Rα expression on the surface of total population of myeloid cells (CD45⁺CD11b⁺ cells) and different subsets of myeloid cells, as well monocytic MDSCs (CD11c″ CD11B⁺ Ly6G⁻ Ly6C⁺), polymorphonuclear MDSCs (CD11c⁻CD11B⁺ Ly6G⁺ Ly6C⁻), TAMs (CD45⁺ CD11B⁺ F4/80^{hi} Ly6C⁻), B cells (CD45⁺ CD19⁺), and dendritic cells (CD45⁺ CD11b^{low} CD11c^{hi}) harvested from brains and splenocytes of mice bearing intracranial CT2A and GL261 murine glioma tumors, n=4. Paired t-test. ***p<0.001. E. CD8+ T cells proliferate in the presence of recombinant murine IL15 in co-culture with wild type MDSC, but not with IL15Rα KO MDSCs or absence of IL15 (negative control). A representative flow plot showing proliferation of Cell Trace Violet labeled CD8+ T cells at 1:32 ratio of MDSCs to T cells (red) or negative control (blue; unstimulated). Quantitative analysis of T cells proliferation in co-cultures are shown. CD8+ T cells activated with activating beads served as a positive control in the proliferation assay.

FIGS. 3A-3G. CAR T cells modified to express IL15 successfully kill glioma cells in vitro. A. CAR.Δ. CAR, CAR IL15s and CAR.IL15f murine constructs were designed as described in the Examples section. B. Schematic diagram shows potential interaction of CAR.IL15s and CAR.IL15f with glioma and cells of the tumor microenvironment. C. Modeling of antigen-binding part of CAR protein, a scFvIL13Rα2 (clone 47) alone and as a fusion with IL15. D. Flow cytometry analysis to quantify CAR T cell transduction efficiency using a fluorochrome conjugated antibody against Thy1.1. E. Trypan blue exclusion assay was performed to determine viability of CAR, CAR.IL15s, and CAR.IL15f T cells. Values presented as % change in viability over 5 days post activation. F. Flow cytometry analysis of IL15 expression on the surface of CD8⁺ Thy1.1+ CAR T cells (n=3, ***p<0.001), and analysis of the CAR T cells for their memory phenotype after vital transduction following two days of rest after retroviral transduction in vitro (n=3, *p<0.05). G. Analysis of the viability of parental and IL13Rα2-expressing GL261 and CT2A cells in co-culture with CAR T cells. Non-transduced (NT) T cells were used as a negative control (n=3, ***p<0.001). Viability values in each group were normalized to NT cells. The experiment was repeated two times.

FIGS. 4A-4E. CAR T cells modified to express IL15 kill MDSC and modulate their immunosuppressive phenotype. A. Experimental setup of co-cultures of bone-marrow-derived MDSC and CAR T cells in vitro. B. Comparison of cytotoxic activity of CAR T cells towards MDSC (n=3, *p<0.05, ***p<0.001). C-E. Flow cytometry analysis of MDSC from co-cultures with CAR T cells for production of IL10, Arginase, and TGFβ (n=3, **p<0.01, ***p<0.001). The experiment was repeated two times.

FIGS. 5A-5E. CAR-IL15f T cells are superior to CAR.IL15s and conventional CAR T in mediating survival of mice in syngeneic models of glioma. A. Survival analysis of mice bearing CT2A-IL. 13Rα2 glioma and treated with 1×10⁶ of CAR T cells (n=10, *p<0.05). B. Mice surviving long-term were re-challenged with parental CT2A line. C. Survival analysis of mice bearing GL261-IL13Rα2 glioma and treated with 1×10⁶ of CAR T cells (n=10, *p<0.05). D. Mice surviving long-term were re-challenged with parental G1261 line. E. Morphometric analysis of tumor area in the brain of mice bearing GL261-IL13Rα2 glioma tumors 10 days after the treatments of CAR T cells (n=5/group), and representative images of H&E stain of the brain of mice after CAR T cell treatment.

FIGS. 6A-6D. IL15-modified CAR T cells alter the tumor microenvironment in murine glioma. A. (i) Frequency of adoptively transferred proliferating (CTV-labeled) CAR T cells in the brain of mice bearing GL261-IL13Rα2 tumors 7 days after CAR T cells treatment, (ii-iii) CD3⁺, CD8⁺ T cells, and (iv) CD8⁺ T cells expressing markers of adoptive memory. B. Frequency of host (i) CD45 CD8″ T cells, (ii) NK cells, (iii) B cells, and (iv) CD11b⁺ TAMs in the brain of mice bearing GL261-IL13Rα2 tumors 7 days after CAR T cells treatment. C. Immunostaining of tumor tissue sections for CD8⁺ T cells, B cells, and CD11b⁺ cells in PBS, CAR, and CAR.IL15f groups. D. Double stain for B cells and CD3⁺ T cells in the area of B cells' accumulation on the tumor/brain border.

FIGS. 7A-7C. Analysis of IL15Rα distribution in tissues of patients. A. The analysis of the spatial distribution of IL15Rα mRNA in GBM tissue in the IvyGap RNAseq data set shows the preferential expression of IL15Rα in the area of microvascular proliferation. ***p<0.001. B. Spearman Rank Correlation analysis of GBMs from TCGA (n=156) and CGGA (n=225) of RNA-seq data sets shows that IL15Rα expression correlates with expression of MRC1 and TGFB1, markers of suppressive (M2-like) macrophages. C. Gating strategy used for flow cytometric analysis of IL 15Rα expression in immune cell compartment from matched patient tumor and PBMC samples represented in two separate panels. ***p<0.001.

FIGS. 8A-8D Analysis of IL15Rα expression by human and murine glioma cells. A. Histological analysis of human GBM tissues (n=10) for expression of IL15Rα. Representative images of tissues with negative and positive expression for IL15Rα expression. B. (i) RT-qPCR and flow cytometry analysis (ii and iii) of murine GL261 and CT2A glioma lines for IL15Rα expression. C. (i) RT-qPCR and flow cytometry analysis (ii and iii) of GBM39, GBM6, and GBM12 PDX lines for IL15Rα expression. D. Representative gating from of IL12Rα expression on the surface of total population of myeloid cells in brain and spleen of tumor bearing animals.

FIGS. 9A-9D. Interaction of CD163 and glioma cells in glioma tissue, Viability of CAR T Cells post transduction, and T Cell transduction gating strategy. A. Multiplex staining of GBM patient tissues to identify a fraction of CD163⁺ cells, representing macrophages, and Sox2⁺ cells, representing glioma cells. B. Quantitative analysis of 21 patient samples shows that CD163⁺ and Sox2⁺ cells are found in immediate proximity to each other within the tumor bed. C. Trypan blue exclusion assay was performed to determine expansion profiling of various CAR T Cell constructs 5 days post activation. Values presented % change in total live cells. D. Gating strategy to analyze CAR, CAR. IL15s, and CAR.IL12f transduction efficiency.

FIGS. 10A-10D. RNAseq analysis of tumors treated with IL15-modified CAR T cells reveals a difference in the expression of immunosuppressive factors. A. Experimental setup for purification of CD45⁻ cells from glioma tumors and bulk RNA sequence analysis. B. The volcano plot depicts changes in the expression of genes in response to treatment with CAR.IL15s. Top hits are shown in the plot. C. GO pathway analysis of microenvironment after CAR and CAR.IL15s T cells treatments. D. Changes in the expression of genes associated with the immunosuppressive phenotype of MDSC in tumor microenvironment seven days after treatment with CAR and CAR.IL15s T cells (n=3, p<0.05)

FIGS. 11A-11B. Histological analysis of the brain collected from mice surviving a long-term after CAR T cells treatment. A. H&E (i) and CD8⁺ T cells (ii) stain of tissue sections from mouse surviving long-term and re-challenged with parental CT2A glioma line; (iii). Quantitative analysis of CD8⁺ T cells in the brain. B. H&E (i) and CD8⁺ T cells (ii) stain of tissue sections from mouse surviving long-term and re-challenged with parental Gl261 glioma line; (iii). Quantitative analysis of CD8+ T cells in the brain.

FIGS. 12A-12B. Analysis of immune cells in spleens of mice bearing GL261-IL13Rα2 and treated with CAR T cells. A. Analysis of spleens from mice treated with CAR T cells for frequency of CD8+ T cells, NK cells, B cells, and CD11b+ MDSCs. N=5/group. B. Gating strategy for flow cytometric analysis of CD45+ immune cells isolated from GL261 tumor-bearing mice 7 days post-treatment with CAR T cells. Figure represents three separate flow panels used.

FIG. 13. CAR.IL15f T cells improve survival of mice bearing CT2A-IL13Rα2 tumors treated with whole brain irradiation (XRT) and temozolomide therapy (TMZ). Mice bearing intracranial CT2A-IL13Rα2 were treated with 3Gy irradiation for 3 days (total 9Gy) followed by TMZ at 50 mg/kg for 5 consecutive days via i.p. route. Three days after the last doze of TMZ, mice were treated systemically (i.v. route) with 10×10⁶ non-transduced T cells (NT, n=9)), CAR (n=10) or CAR.IL15f T cells (n=10). Log-rank test was used to evaluate the significance in survival between groups.

### DETAILED DESCRIPTION

The present disclosure provides fusion proteins comprising chimeric antigen receptors (CARs) comprising a single chain antibody to IL13Rα2 (scFv) and interleukin (IL15), polynucleotides encoding them, and methods of their use. The fusion proteins are useful for improving CAR T cell targeting to tumor cells and tumor associated macrophages. It is to be understood that the disclosed compositions are intended for use in any procedure where IL15-modified CAR T cells is desired or intended.

In an aspect, provided herein is an engineered polynucleotide encoding a fusion protein comprising a chimeric antigen receptor (CAR) comprising a single chain antibody to IL13Rα2 (scFv) and modified with interleukin 15 (IL15). The fusion protein as a dual-targeting agent against glioma and suppressive tumor microenvironment cells. It also modulates the tumor microenvironment by enhancing the proliferation of CAR T cells themselves, inviting the infiltration of the host's CD8 T cells, natural killer (NK) cells, and B cells while decreasing the frequency of CD11b (tumor-associated macrophages) and myeloid-derived suppressor cells (MDSCs), and modulating the immunosuppressive function of MDSCs within the tumor microenvironment. The IL15 may be an active portion of IL15 or the full protein. The IL15 may be human IL15. The IL15 may be humanized IL15. The engineered polynucleotide may encode a fusion protein comprising a human IL15 and a human chimeric antigen receptor comprising a single chain antibody to IL13Rα2 (scFv). The engineered polynucleotide may comprise a sequence of SEQ ID NO: 1 or 27 or at least 95% identity to SEQ ID NO: 1 or 27. The fusion protein encoded by the engineered polynucleotide may comprise SEQ ID NO: 2, 26, or 28 or a sequence having at least 95% identity to SEQ ID NO: 2, 26, or 28.

The term “polynucleotide” is used herein interchangeably herein with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, including DNA, RNA, ORFs, analogs and fragments thereof. The polynucleotides disclosed herein may be optimized, for example codon optimized or host cell optimized.

The terms “engineered polynucleotide”, “recombinant polynucleotide”, “genetically engineered polynucleotide”, and “genetically modified polynucleotide” may be used interchangeably and refer to any manipulation of a polynucleotide that results in a detectable change in the polynucleotide, wherein the manipulation includes, but is not limited to, any changes in sequence of the naturally occurring polynucleotide or inclusion of non-naturally occurring nucleotides or nucleosides.

As used herein, the terms “protein” and “polypeptide” are used interchangeably herein to designate a series of amino acid residues connected to by peptide bonds between the alpha-amino and carboxy groups of adjacent residues. The terms “protein” and “polypeptide” refer to a polymer of protein amino acids, including modified amino acids (e.g., phosphorylated, glycated, glycosylated, etc.) and amino acid analogs. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing.

Provided herein is a fusion protein comprising a chimeric antigen receptor (CAR) comprising a single chain antibody to IL13Rα2 (scFv) and modified with interleukin 15 (IL15), as encoded by the polynucleotides disclosed herein. As used herein a “fusion protein” refers to proteins created through the joining of two or more genes that originally coded for separate proteins (e.g., as a fusion or as separate chains linked by one or more disulfide bonds, etc.). Translation of the two joined genes results in a single polypeptide with functional properties derived from each of the original proteins.

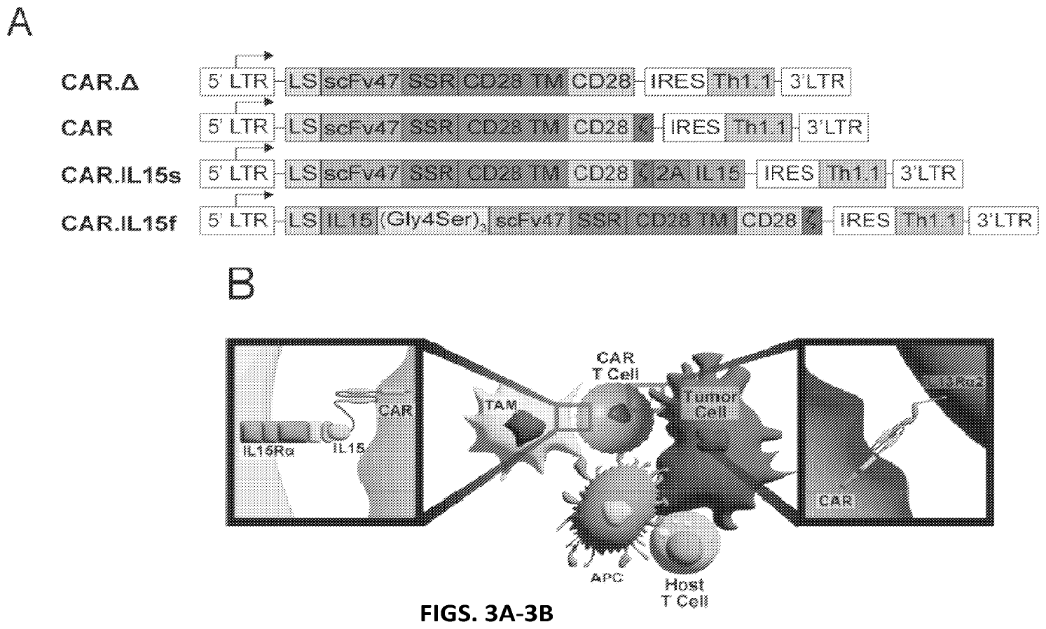

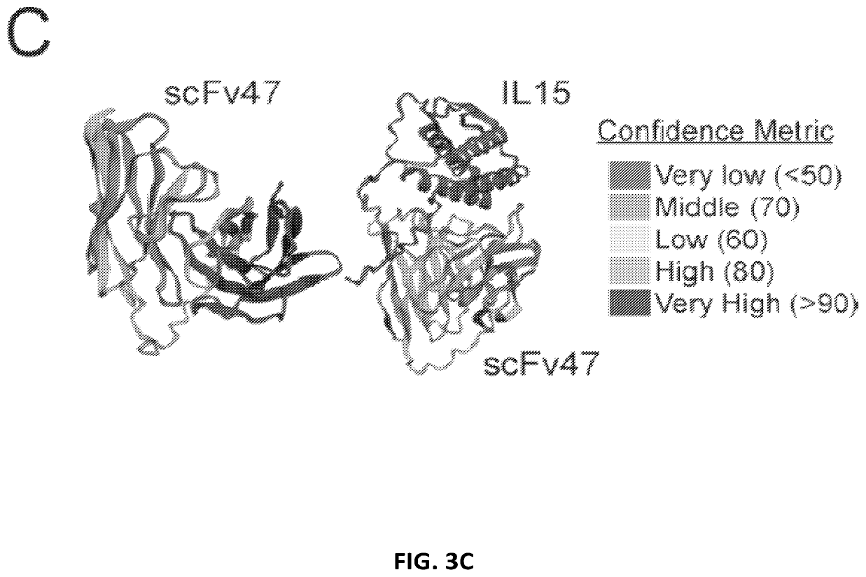

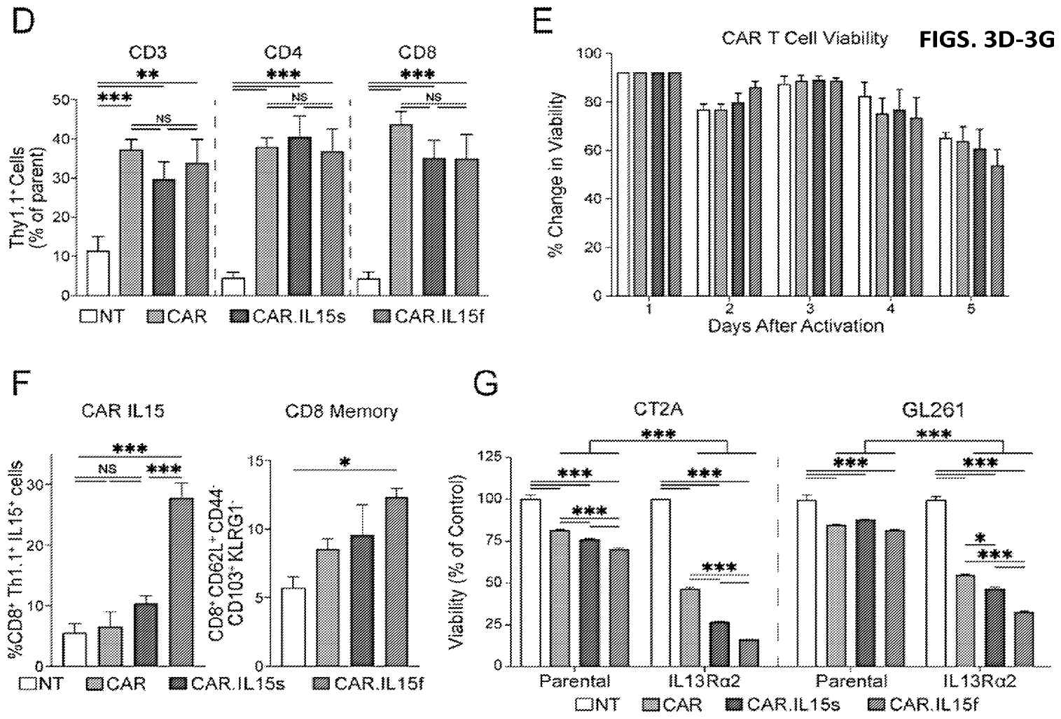

The terms “chimeric antigen receptor” (“CAR”), “chimeric T cell receptor”, “artificial T cell receptor”, and “chimeric receptor” refer to a polypeptide that binds to cell membrane and has a pre-defined binding domain operably linked to an intracellular signaling domain, where the binding domain has specificity to a target antigen operably and the signaling domain activates the cell when the antigen is bound. More particularly, CARs are engineered receptors, which graft an antigen specificity onto a cytotoxic cell, for example T cells, NK cells or macrophages. For example, CAR proteins are engineered to give T cells the new ability to target a specific protein. This particularly includes receptors wherein the extracellular domain and the cytoplasmic domain are not naturally found together on a single receptor protein. Further, the chimeric receptor is different from the T cell receptor expressed in the native T cell lymphocyte. The CARs of the present invention may comprise an extracellular domain with at least one antigen specific targeting region, a transmembrane domain (TM), and an intracellular domain (ID) including one or more co-stimulatory domains (CSD) in a combination that is not naturally found together on a single protein (exemplary constructs are found in FIG. 3A).

An extracellular domain is external to the cell or organelle and functions to recognize and respond to a ligand. A transmembrane domain spans the membrane of a cell. An intracellular domain is situated inside a cell. Intracellular co-stimulatory domains provide secondary signals to the cell. They can recruit signaling molecules, cytoskeletal mobilization or induce cell proliferation, differentiation or survival. In the present disclosure a CAR may include an antigen specific extracellular domain, a transmembrane domain and one or more intracellular domains with one or more co-stimulatory domains.

The extracellular domain antigen binding region of the present disclosure comprises a single chain variable fragment (scFv) which is comprised of six complementarity determining regions (CDRs). CDRs are hypervariable domains that determine specific antibody binding. scFv are polypeptides that contain the variable light chain and variable heavy chain of an antibody connected by a flexible linker peptide. The scFv of the present disclosure may comprise the scFv of IL13Rα2 (scFv). The scFv may be clone 47 (scFv47). The scFv may be encoded by a polynucleotide sequence comprising SEQ ID NO: 6, 22 or a sequence having at least 95% identity to SEQ ID NO: 6, 22. The scFv may comprise SEQ ID NO: 7, 23, 31, 32, 33, 34, or 35 or a sequence having at least 95% identity to SEQ ID NO: 7, 23, 31, 32, 33, 34, or 35.

The CAR of the present disclosure may comprise a transmembrane domain and a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety and the transmembrane domain. The hinge sequence can be any suitable sequence derived or obtained from any suitable molecule. For example, the hinge sequence may be derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD154, KIRDS2, OX40, CD2, CD27, LFA-1 (CD11 a, CD18), ICOS (CD278), 4-1 BB (CD137), GITR, CD40, BAFFR, HVEM (LIGHTR), SLAMF7, NKp80 (KLRF1), CD19, IL2R beta, IL2R gamma, IL7R a, ITGA1, VLA1, CD49a, ITGA4, IA4, CD49D, ITGA6, VLA-6, CD49f, ITGAD, CD 11 d, ITGAE, CD103, ITGAL, ITGAM, CD11 b, ITGAX, CD11 c, ITGB1, CD29, ITGB2, ITGB7, TNFR2, DNAM 1 (CD226), SLAMF4 (CD244, 2B4), CD84, CD96 (Tactile), CEACAM 1, CRTAM, Ly9 (CD229), CD160 (BY55), PSGL1, CD 100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively, the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR. In some embodiments, the CAR has more than one transmembrane domain, which can be a repeat of the same transmembrane domain or can be different transmembrane domains.

The CAR of the present disclosure may comprise at least one intracellular signaling domain. The signal sequence plays a determinant role in protein distribution and can allow the CAR to be glycosylated and anchored in the cell membrane. The intracellular signaling domain may be a co-stimulatory domain. A costimulatory domain is required for an efficient antigen response in immune cells. The intracellular signaling domain may be derived from CD3 zeta (CD3ζ (TCR zeta, GenBank acc no. BAG36664.1). T-cell glycoprotein CD3 zeta (CD3ζ chain, also known as T-cell receptor T3 zeta chain or CD247 (Cluster of Differentiation 247), is a protein that in humans is encoded by the CD247 gene. Other co-stimulatory domains include CD28, 4-1BB, OX-40. ICOS and other members of the TNF receptor superfamily or immunoglobulin (lg) superfamily. In exemplary embodiments, the CAR comprises a CD28 and CDζ co-stimulatory domain. In other embodiments, the CAR comprises at least one of a CD28 and a 4-1BB co-stimulatory domain. However, any co-stimulatory domains may be used. For example, the IL15.CAR may have at least 95% identity to SEQ ID NO: 36 or 37 (IL15 and scFv IL13Rα2), and include any co-stimulatory domain. Members of the TNF superfamily form trimeric structures, and their monomers are composed of beta-strands that orient themselves into a two-sheet structure. The TNF superfamily ligands include lymphotoxin alpha, tumor necrosis factor, lymphotoxin beta, OX40 ligand, CD40 ligand, Fas ligand, CD27 ligand, CD30 ligand, CD137 ligand, TNF-related apoptosis-inducing ligand, receptor activator of nuclear factor kappa-B ligand, TNF-related weak inducer of apoptosis, a proliferation-inducing ligand, B-cell activating factor, LIGHT, vascular endothelial growth factor, TNF superfamily member 18 and ectodysplasin A. These ligands then bind to receptors in the TNF superfamily. Other co-stimulatory domains include lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2. Any of the aforementioned co-stimulatory domains or others may be used in isolation or in any combination in the CARs disclosed herein.

“Percentage of sequence identity”, “percent similarity”, or “percent identity” is determined by comparing two optimally aligned sequences over a comparison window, wherein the portion of the polynucleotide or peptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantial similarity” of polynucleotide or peptide sequences means that a polynucleotide or peptide comprises a sequence that has at least 75% sequence identity. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

Soluble IL15 is known to be unstable unless it exists in a complex with IL15Rα. By fusing IL15 to the scFv portion of the IL13Rα2 CAR molecule, the inventors were able to generate a more stable source of IL15. When T cells express the fusion protein, IL15 is constitutively expressed on the surface of the cells. The IL15 may be encoded by a polynucleotide sequence comprising SEQ ID NO: 3 or 19 or a sequence having at least 95% identity to SEQ ID NO: 3 or 19. The IL15 may be encoded by a polypeptide sequence comprising SEQ ID NO: 4, 20, or 30 or a sequence having at least 95% identity to SEQ ID NO: 4, 20, or 30. The IL15 may be fused to the scFv via a linker. The linker keeps each of the variable regions at a distance that favors proper folding and formation of the antigen-binding site while also minimizing oligomerization of the scFv. A simple form of a linker is the hinge region of IgG1. The linker may be a flexible linker. The linker may be 10-25 amino acids long and made up of glycine and serine amino acids, and optionally with dispersed hydrophilic residues to increase solubility. The linker may be (Gly₄S)₃. The IL15 may also include a signal peptide or leader sequence (LS) for traversing the cell membrane and expressing the IL15 efficiently. The signal peptide/leader sequence may comprise SEQ ID NO: 38. However, any signal peptide known in the art may be used.

In a second aspect, provided herein is an engineered polynucleotide encoding (a) a chimeric antigen receptor, the chimeric antigen receptor comprising a single chain antibody to IL13Rα2 (scFv), and (b) a secretory interleukin 15 (IL15s). The resulting secreted IL15s increases adoptive T cell proliferation, decreases MDSCs, and modulates MDSC secretion of immunosuppressive molecules, albeit to a lesser extent than the IL13Rα2 (scFv)/IL15 fusion protein. The IL15s may be an active portion of IL15 or the full protein. The IL15 may be human IL15. The IL15 may be humanized IL15. In the protein encoded by the engineered polynucleotide, the CAR and the IL15 may be connected via a self-cleavage site. Self-cleavage sites are known in the art and include a 2A self-cleaving peptide. The self-cleavage site may be a T2A self-cleaving peptide. The self-cleavage site may comprise SEQ ID NO: 18 or a sequence having at least 95% identity to SEQ ID NO: 18. The engineered polynucleotide may comprise SEQ ID NO: 8 or a sequence having at least 95% identity to SEQ ID NO: 8. The encoded protein may comprise the polypeptide sequence of SEQ ID NO: 9 or a sequence having at least 95% identity to SEQ ID NO: 9. The engineered polynucleotide may comprise a sequence encoding any of the scFv47, CD28 and CD3zeta, IL15, and cleavage peptides described herein.

Any of the engineered polynucleotides described herein may be incorporated into a construct. As used herein, the term “construct” refers to a recombinant polynucleotide, i.e., a polynucleotide that was formed artificially by combining at least two polynucleotide components from different sources (natural or synthetic). For example, the constructs may comprise a portion of the coding region of a transgene of interest (e.g. a single chain antibody to IL13Rα2 (scFv), IL15, and IL15s, and portions thereof) operably linked to a promoter that (1) is associated with another gene found within the same genome, (2) is from the genome of a different species, or (3) is synthetic. The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

As used herein, the terms “heterologous promoter,” “promoter,” “promoter region,” or “promoter sequence” refer generally to transcriptional regulatory regions of a gene, which may be found at the 5′ or 3′ side of a polynucleotides described herein, or within the coding region of said polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase. Heterologous promoters useful in the practice of the present invention include, but are not limited to, constitutive, inducible, temporally-regulated, developmentally regulated, chemically regulated, tissue-preferred and tissue-specific promoters. The heterologous promoter may be a plant, animal, bacterial, fungal, or synthetic promoter. Suitable promoters are known and described in the art. Suitable promoters include the T3, T7 and SP6 promoter sequences, which are often used for in vitro transcription of RNA. In mammalian cells, typical promoters include, without limitation, promoters for Rous sarcoma virus (RSV), human immunodeficiency virus (HIV-1), cytomegalovirus (CMV), SV40 virus, as well as the translational elongation factor EF-1α promoter or ubiquitin promoter.

Constructs can be generated using conventional recombinant DNA methods. Constructs may be part of a vector. When referring to a nucleic acid molecule alone, the term “vector” is used herein to describe a nucleic acid molecule capable of transporting another nucleic acid to which it is linked. In contrast, the term “viral vector”, “AAV vector”, or “rAAV vector” is used to describe a virus particle that is used to deliver genetic material (e.g., the constructs) into cells.

The constructs may be expressed in a cell. In some cases, a viral or plasmid vector system is employed for delivery of the constructs. The vector may be a viral vector, and preferably a retroviral vector. The vector may be a lenti-, baculo-, or adeno-viral/adeno-associated viral vector, but other means of delivery may be used (such as yeast systems, microvesicles, gene guns/means of attaching vectors to gold nanoparticles).

The viral or plasmid vectors may be delivered via liposomes, nanocarriers, exosomes, microvesicles, or a gene-gun. A “nanocarrier” as used herein typically has at least one dimension in the 1-500 nanometer scale. A nanocarrier is nanomaterial being used as a transport module for another substance, such as a drug. Commonly used nanocarriers include micelles, polymers, carbon-based materials, nanoparticles, dendrimers, and polymeric or lipid-based carriers like liposomes. The nanocarriers may be exosomes. Exosomes are membrane-bound extracellular vesicles that are produced in the endosomal compartment of most eukaryotic cells. The nanocarrier may be functionalized with the engineered polypeptides described herein. Functionalization enhances the properties and characteristics of nanoparticles through surface modification, and improves their function including biocompatibility and cellular internalization. Nanocarriers have been functionalized with a variety of ligands such as small molecules, surfactants, dendrimers, polymers, and biomolecules.

The engineered polynucleotides, fusion proteins, and proteins described herein may be expressed in a host cell. As used herein, a “host cell” is the cell in which expresses the polynucleotide or polypeptide. The host cell may be a mammalian cell. The host cell may be a human cell. The host cell may comprise an immune cell. The host cell may comprise a T cell, a NK cell or a B cell. The host cell expressing the fusion protein, polynucleotide, or polypeptide may be a CAR-T cell, CAR-NK cell, CAR-B cell or CAR-macrophage. The host cell may further comprise or expresses a cancer therapeutic.

The engineered polynucleotides, fusion proteins, proteins, and host cells may be prepared in a pharmaceutical composition. As used herein, the term “pharmaceutical composition” refers to a chemical or biological composition suitable for administration to a mammal. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions. Examples of compositions appropriate for such therapeutic applications include preparations for parenteral, subcutaneous, transdermal, intradermal, intramuscular, intracoronarial, intramyocardial, intraperitoneal, intravenous or intraarterial (e.g., injectable), or intratracheal administration, such as sterile suspensions, emulsions, and aerosols. In some cases, pharmaceutical compositions appropriate for therapeutic applications may be in admixture with one or more pharmaceutically acceptable excipients, diluents, or carriers such as sterile water, physiological saline, glucose or the like.

In a third aspect, a method of treating a cancer in a subject is provided, the method comprising administering to the subject a therapeutically acceptable amount of the pharmaceutical compositions described herein. Cancer is a group of diseases involving abnormal cell growth with the potential to invade or spread to other parts of the body. For example, treating cancer in a subject includes the reducing, repressing, delaying or preventing cancer growth, reduction of tumor volume, and/or preventing, repressing, delaying or reducing metastasis of the tumor. Treating cancer in a subject also includes the reduction of the number of tumor cells within the subject. The term “treatment” can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; (d) reducing or ameliorating at least one symptom of cancer; and (e) extending the survival of the subject. In some embodiments, the optimum effective amount can be readily determined by one skilled in the art using routine experimentation. Cancer treatment includes, but is not limited to chemotherapy, radiation, bone marrow transplant, surgery and immunotherapy. In some embodiments, the cancer is glioblastoma.

In a fourth aspect, altering a tumor microenvironment in a subject is provided, the method comprising administering to the subject a therapeutically acceptable amount of the pharmaceutical compositions described herein. Altering the tumor environment may include reducing the amount or concentration of myeloid-derived suppressor cells in the tumor microenvironment; reducing the levels of at least one of IL10, arginase 1, and TGF-β; and/or increasing the frequency of NK cells and B cells in the tumor microenvironment. Altering the tumor microenvironment may increase the anti-tumor response. The tumor microenvironment is the environment around a tumor, including the surrounding blood vessels, immune cells, fibroblasts, signaling molecules and the extracellular matrix. The anti-tumor response is the innate and adaptive immune response which lead to tumor control. Myeloid-derived suppressor cells are immature myeloid cells that are characterized by the ability to suppress immune responses and expand during cancer, infection, and inflammatory diseases.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

Formulations may be designed or intended for oral, rectal, nasal, systemic, topical or transmucosal (including buccal, sublingual, ocular, vaginal and rectal) and parenteral (including subcutaneous, intramuscular, intravenous, intraarterial, intradermal, intraperitoneal, intrathecal, intraocular and epidural) administration. In general, aqueous and non-aqueous liquid or cream formulations are delivered by a parenteral, oral or topical route. In other embodiments, the compositions may be present as an aqueous or a non-aqueous liquid formulation or a solid formulation suitable for administration by any route, e.g., oral, topical, buccal, sublingual, parenteral, aerosol, a depot such as a subcutaneous depot or an intraperitoneal or intramuscular depot. In some cases, pharmaceutical compositions are lyophilized. In other cases, pharmaceutical compositions as provided herein contain auxiliary substances such as wetting or emulsifying agents, pH buffering agents, gelling or viscosity enhancing additives, preservatives, flavoring agents, colors, and the like, depending upon the route of administration and the preparation desired. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York). For intravenous administration, suitable carriers include physiological saline, bacteriostatic water, Cremophor EL® (BASF, Parsippany, N.J., USA) or phosphate buffered saline (PBS). In all cases, a composition for parenteral administration must be sterile and should be formulated for ease of injectability. The composition should be stable under the conditions of manufacture and storage, and must be shielded from contamination by microorganisms such as bacteria and fungi.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician, the dosing formulation can be provided in a kit containing all necessary equipment (e.g., vials of drug, vials of diluent, syringes and needles) for a course of treatment (e.g., 7 days of treatment).

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

The preferred route may vary with, for example, the subject's pathological condition or age or the subject's response to therapy or that is appropriate to the circumstances. The formulations can also be administered by two or more routes, where the delivery methods are essentially simultaneous or they may be essentially sequential with little or no temporal overlap in the times at which the composition is administered to the subject.

Suitable regimes for initial administration and further doses or for sequential administrations also are variable, may include an initial administration followed by subsequent administrations, but nonetheless, may be ascertained by the skilled artisan from this disclosure, the documents cited herein, and the knowledge in the art.

The terms “effective amount” or “therapeutically effective amount” refer to an amount sufficient to effect beneficial or desirable biological and/or clinical results. The amount of the pharmaceutical composition that is therapeutically effective may vary depending on the particular pathogen or the condition of the subject. Appropriate dosages may be determined, for example, by extrapolation from cell culture assays, animal studies, or human clinical trials taking into account body weight of the patient, absorption rate, half-life, disease severity and the like. The dosage lies preferably within a range of circulating concentrations that include the ED50 with little or no toxicity. The dosage may vary within this range depending upon the dosage form employed and the route of administration utilized. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Miscellaneous

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The invention will be more fully understood upon consideration of the following non-limiting examples.

The present invention has been described in terms of one or more preferred embodiments, and it should be appreciated that many equivalents, alternatives, variations, and modifications, aside from those expressly stated, are possible and within the scope of the invention.

The following Examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

The inventors and others have previously demonstrated that interleukin-13 receptor alpha 2 (IL13Rα2) can serve as a CAR target in both xenografts and syngeneic models of GBM, and IL13Rα2-CAR T cells have been evaluated in early phase clinical studies. However, their activity in clinical studies and in syngeneic GBM models was limited.¹⁴⁻¹⁷ Expressing cytokines, chimeric cytokine receptors, or constitutively active cytokine receptors improve the effector function of CAR T cells in preclinical models.¹⁸⁻²² The inventors and others have demonstrated that these cells have enhanced anti-tumor activity, and early clinical studies are in progress.^{15, 23, 24}

The majority of preclinical studies with IL15-expressing CAR T cells have been conducted in xenograft models, which do not allow studying the effects of IL15 on tumor-infiltrating immune cells. IL15 binds not only the IL2/15Rβ and γC chains expressed in T cells, but is also trans-presented to T cells by binding to IL15 receptor alpha (IL15Rα) expressed on antigen presenting cells. 30, 31 It was previously unknown if myeloid-derived suppressor cells (MDSC), a major component of the GBM TME^{25, 26}, express IL15Rα and can be targeted with CAR T cells.

IL15Rα is Expressed by Cells of the TME at Higher Levels than in Peripheral Blood of GBM Patients.

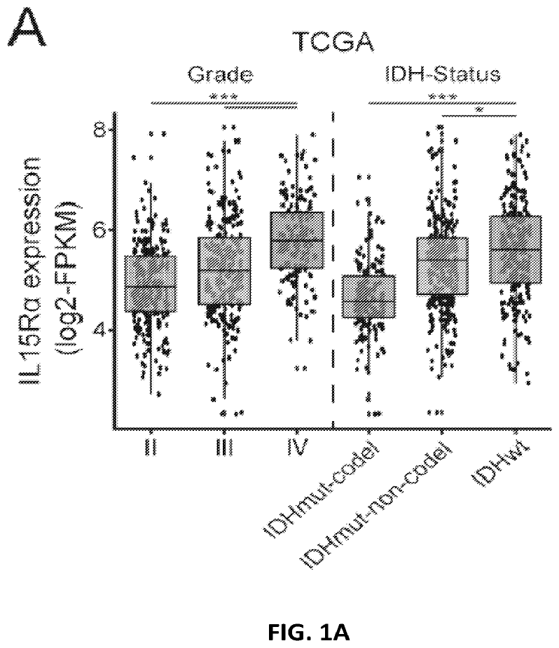

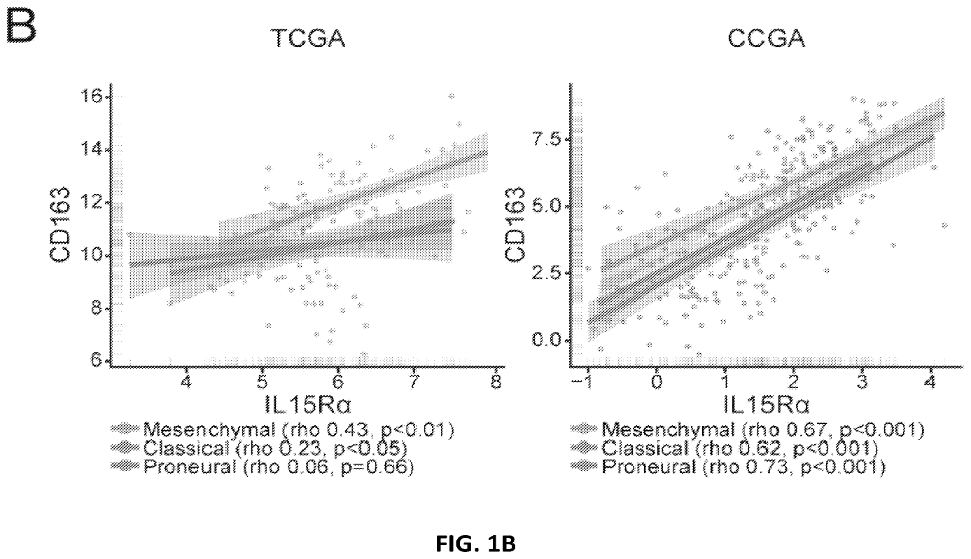

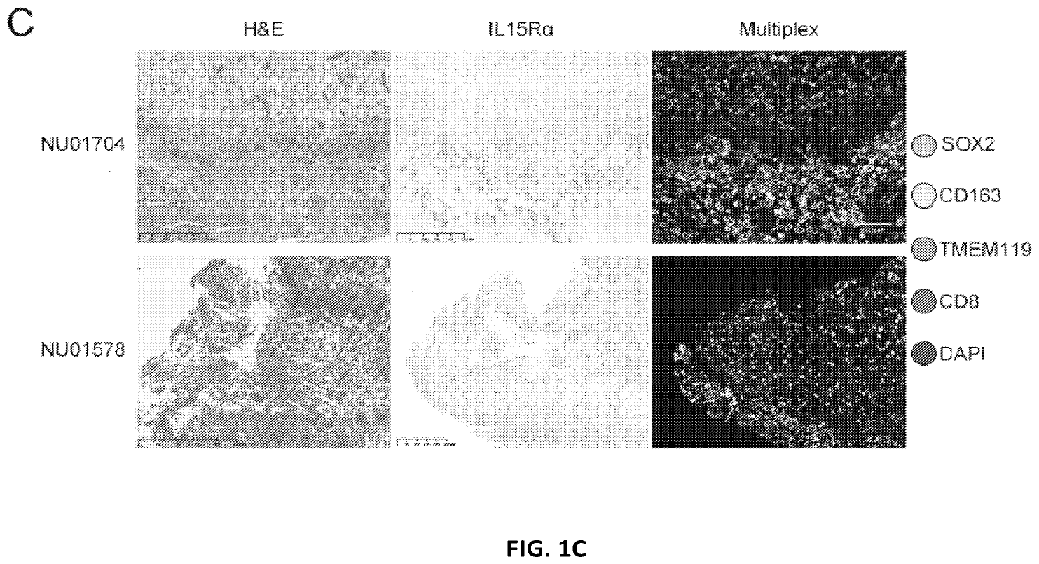

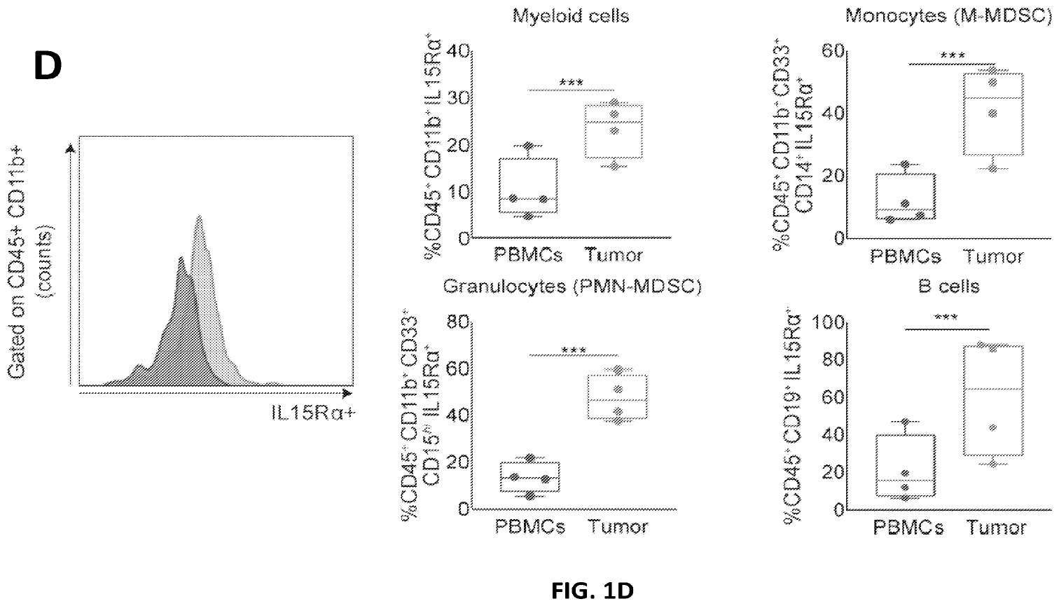

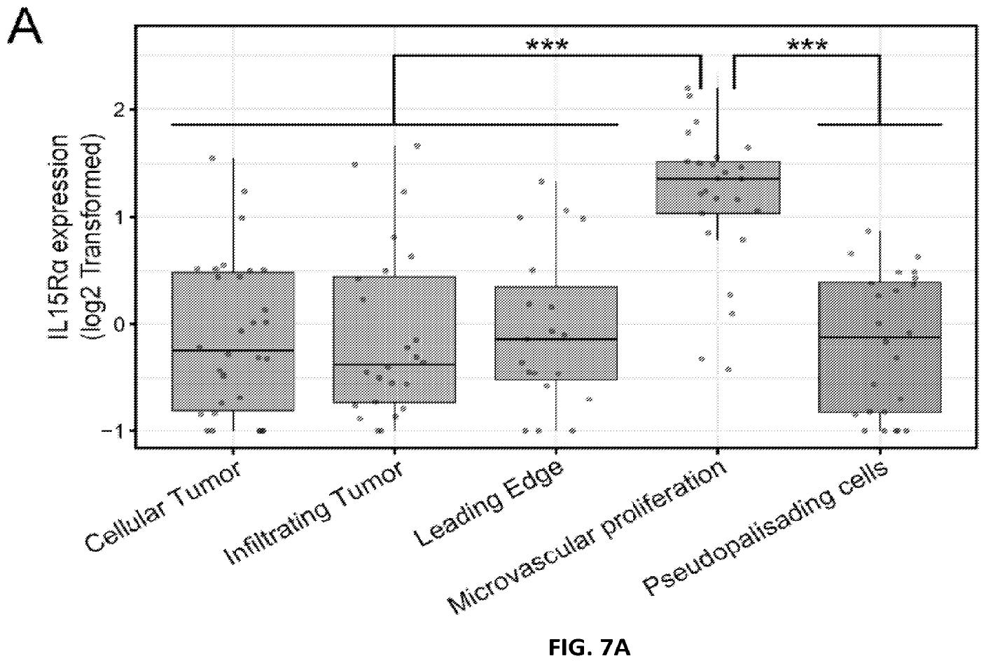

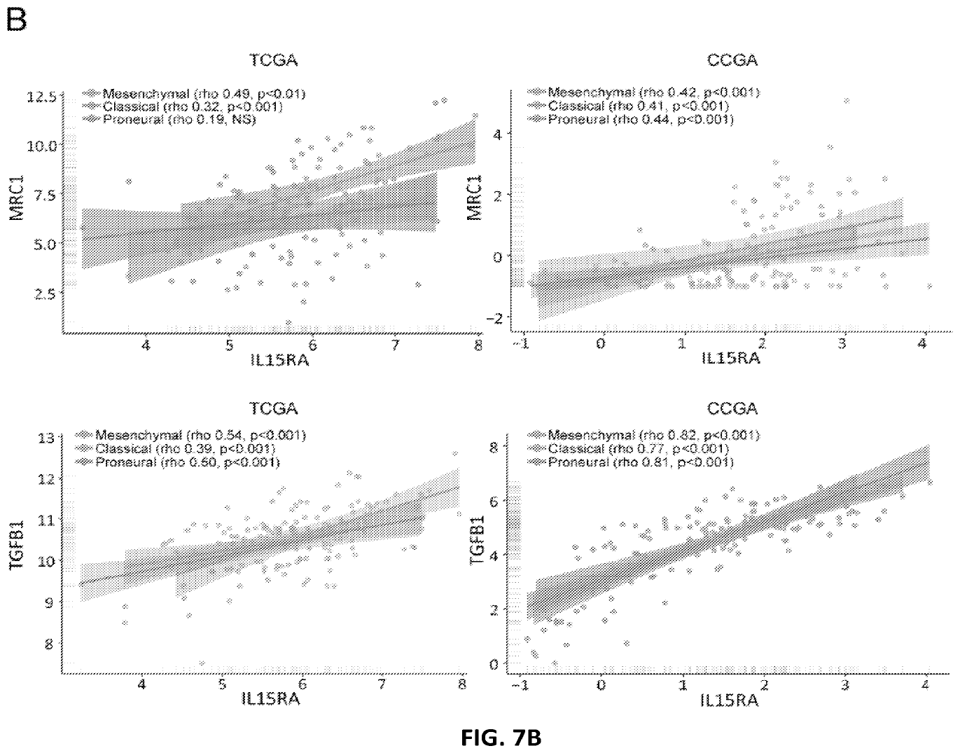

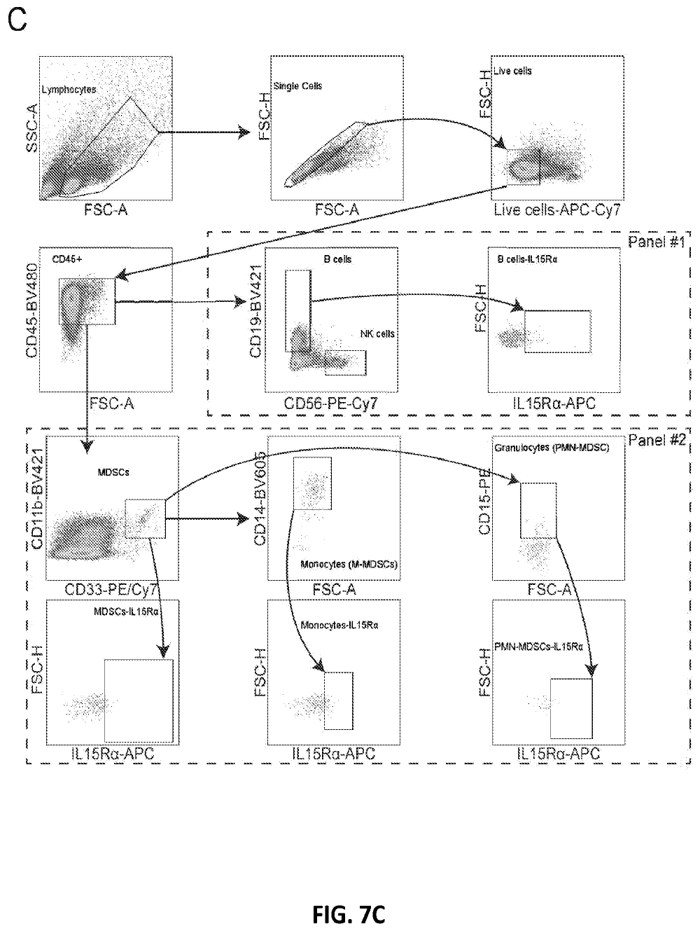

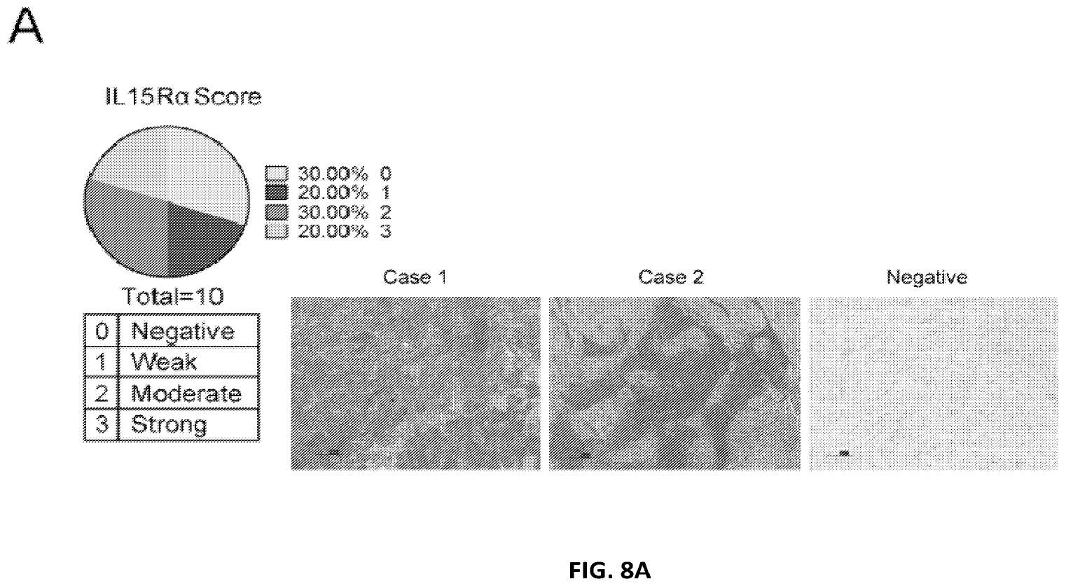

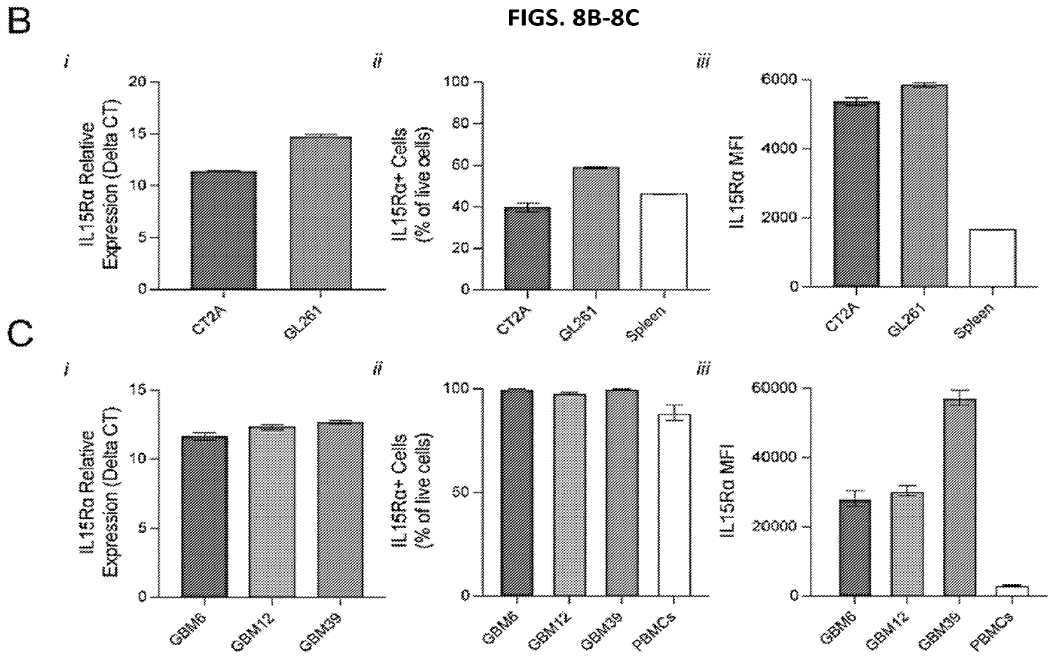

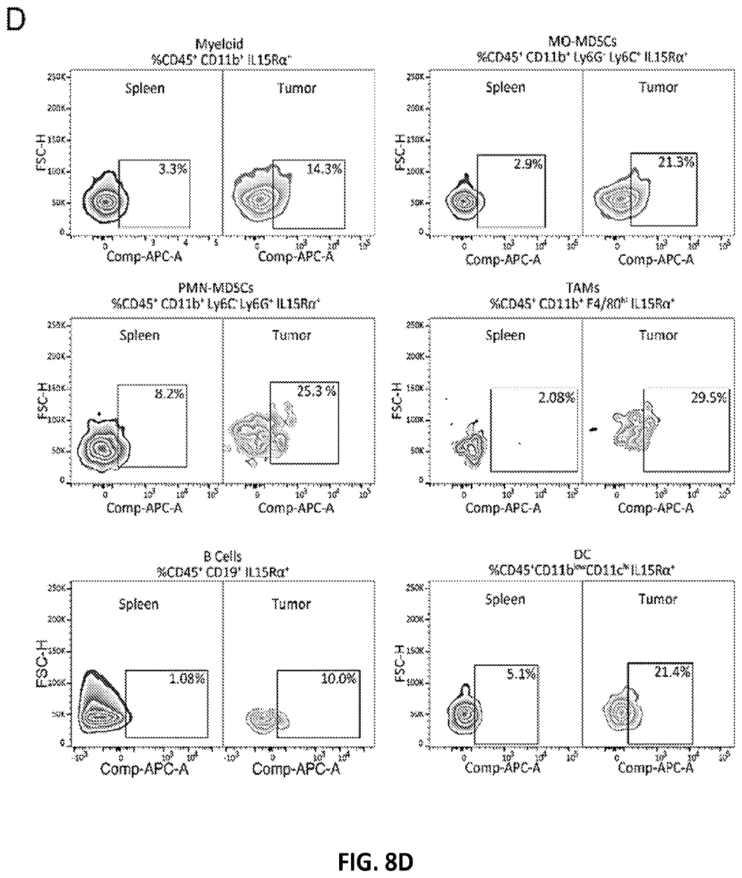

To investigate if myeloid cells express IL15Rα in the GBM TME, the inventors first performed a transcriptomic analysis of GBMs from The Cancer Genome Atlas (TCGA). IL15Rα expression correlates with histological grade, with significantly higher expression seen in grade IV compared to lower grade brain tumors, and positively correlates with Isocitrate Dehydrogenase-wild type (IDHwt) status, with significantly higher expression seen in IDHwt compared to both codel and non-codel IDH mutant tumors (FIG. 1A). Further analysis revealed a positive correlation between IL15Rα and CD163, CD206, and TGFB1 markers associated with M2 phenotype of macrophages³², in both TCGA and Chinese Glioma Genome Atlas (CGGA) data sets (FIG. 1B, FIG. 7B). Analysis of human GBM tissue in the Ivy Glioblastoma Atlas Project (Ivy GAP) revealed higher IL15Rα expression levels in areas of microvascular proliferation and hyperplastic blood vessels of GBM. (FIG. 7A). Immunostaining of tumor tissue sections from GBM patients displayed IL15Rα expression coinciding with macrophage-rich (CD163⁻) regions (FIG. 1C). Analysis of 4 paired peripheral blood and GBM tissue samples revealed a significantly higher IL15Rα expression on the surface of cells in the myeloid compartment (CD45⁺CD11b⁺ cells) of GBM than in peripheral blood (FIG. 1D). Further stratification of CD45⁺ cell subsets showed higher IL15Rα expression in tumor derived monocytic myeloid-derived suppressor cells (M-MDSC; CD45⁺, CD11b⁺, CD33⁺, and CD14⁺), granulocytic MDSCs (PMN-MDSC; CD45⁺, CD11b⁺, CD33⁺, and CD15^{hi}), and B Cells (CD45⁺ and CD19⁺) compared to peripheral blood (FIG. 1D, FIG. 7C). Immunostaining of ten patient samples shows that IL15Rα is also expressed by glioma cells, albeit with great intra and inter-tumor variability (FIG. 8A). RT-qPCR and flow cytometry analysis of three patient-derived and two murine glioma cell lines detected IL15Rα expression at both the mRNA and protein level, respectively (FIG. 8B, C).

### Murine Syngeneic Models of Glioma Recapitulate the Expression IL15Rα Pattern in Human GBM

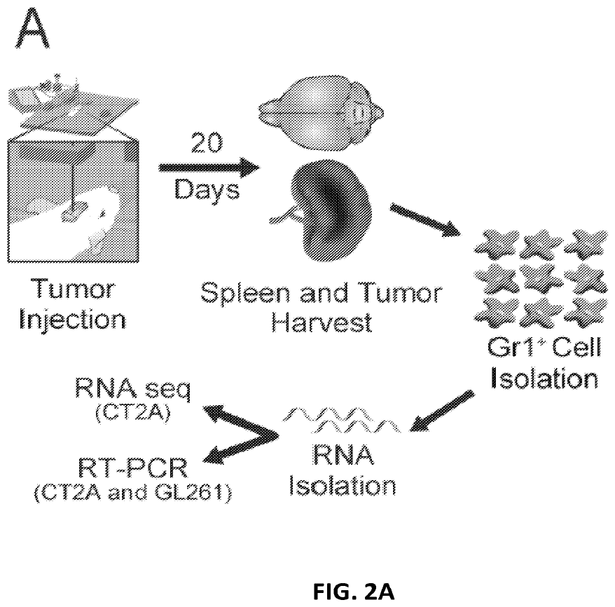

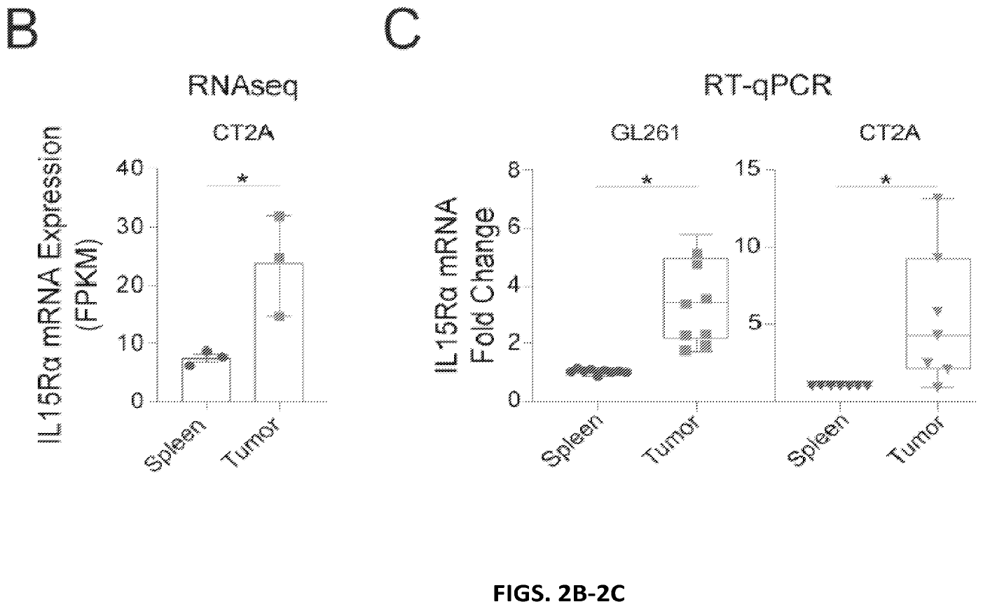

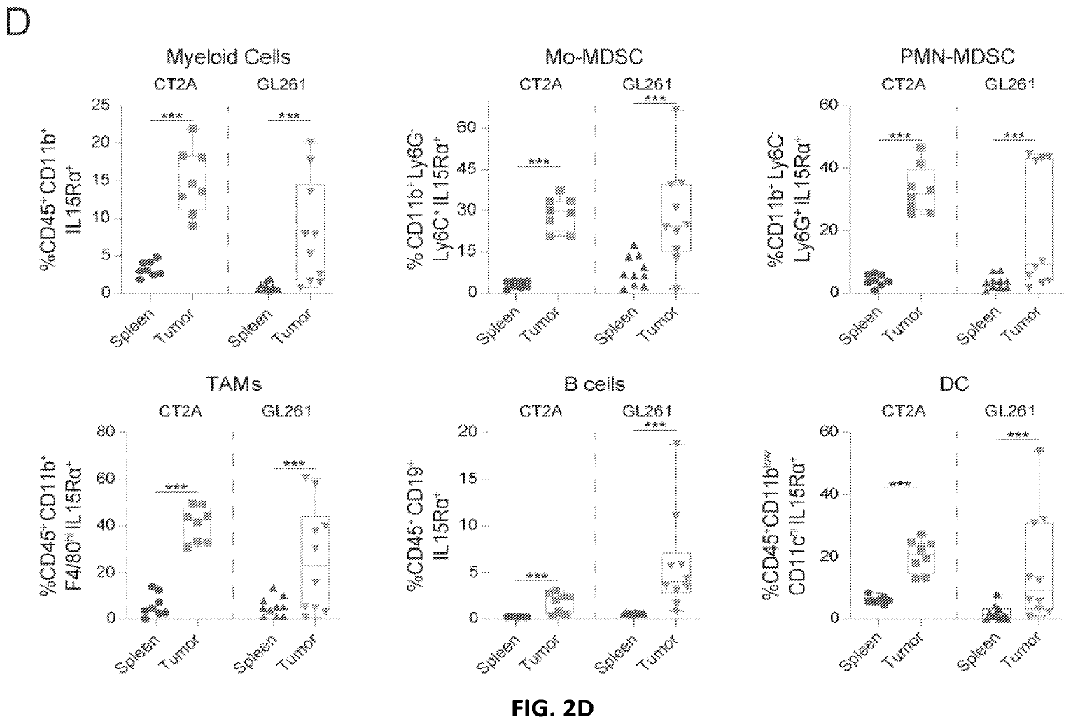

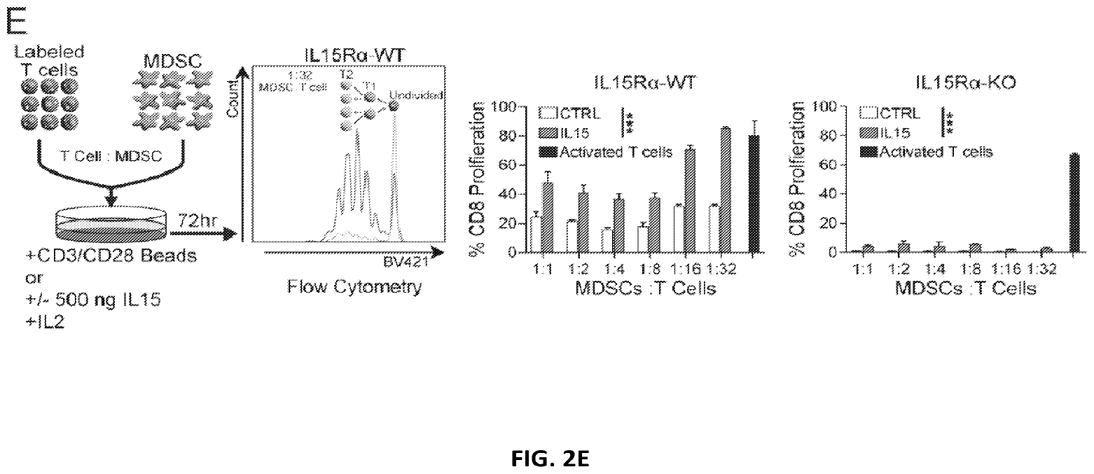

The inventors next sought to determine if the preferential expression of IL15Rα by myeloid cells is recapitulated in murine glioma models. To do so, the inventors isolated Gr1⁺ cells from the tumors and spleens of CT2A and GL261 bearing mice, extracted the mRNA from these cells, and, in separate experiments, performed either bulk RNAseq (PRJNA909275) or RT-qPCR (FIG. 2A). RNA-seq analysis of CT2A bearing mice showed 3.7-fold higher IL15Rα expression in tumor-associated Gr1⁺ cells (log 2-fold change 1.9, n=3, adj. p value=1.19E-10) compared to splenic Gr1⁺ cells (FIG. 2B). Similar results were obtained from RT-qPCR analysis, with 3.5 and 4.2-fold higher IL15Rα expression in tumor-derived cells compared to splenocytes from GL261 and CT2A bearing models, respectively (FIG. 2C). To determine if there are any expression differences in individual cell types, the inventors used flow cytometry to further stratify the immune subsets of CT2A and GL261 bearing animals and compared IL15Rα expression of tumor-derived cells to that of the spleen. The overall tumor-derived myeloid compartment (CD45⁺CD11b⁺) displayed significantly higher IL15Rα expression than myeloid cells from the spleen (FIG. 2D) in both GL261 and CT2A models. Similar results were seen following stratification, with Mo-MDSC (CD45⁺, CD11b⁺, Ly6G⁻Ly6C⁺), PMN-MDSC (CD45⁺, CD11b″, Ly6C;, Ly6G″), and TAMs (CD45⁺, CD11b⁺, F4/80^{hi}), all displaying significantly higher IL15Rα expression differences compared to splenic cells (FIG. 2D). In both CT2A and GL261-bearing animals, the same comparison of B cells (CD45⁺CD19b⁺) and dendritic cells (CD11b⁻CD11c^{hi}) showed significantly higher expression of IL15Rα in the tumor compartment (FIG. 2D; representative flow cytometry plots are shown in FIG. 8D). Taken together, the expression of IL15Rα in these cell types suggests the myeloid compartment may be able to influence T cell activity through IL15 trans-presentation.

To test this, the inventors labeled CD8⁻ T cells with CTV proliferation dye, prepared bone-marrow derived MDSCs from either wild-type (WT) or IL15Rα knock-out (KO) mice, and co-cultured them for 72 h at various ratios with or without recombinant murine IL15 (rIL15), and determined proliferation of T cells via flow cytometry (FIG. 2E)³³. CD3/CD28/CD2-bead activated T cells served as the control for this experiment. In the absence of rIL15, no proliferation of T cells was seen in either condition after 72h of co-culture (FIG. 2E). Following the addition of recombinant rIL15 to cultures, proliferation of CD8⁺ T cells was observed in IL15Rα WT-MDSCs setting, but not IL15Rα KO-MDSC (FIG. 2E).

In summary, murine glioma models follow a similar pattern of IL15Rα expression seen in GBM patients. Furthermore, as shown by the dependency of IL15Rα for IL15 mediated T cell proliferation, the inventors confirmed the ability of IL15Ra expressing MDSCs to trans-present IL15 to CD8⁻ T cells and modulate their activity.

CAR T Cells Armored with IL15 Fusion are More Potent than CAR T Cells Secreting IL15 in Killing IL13Rα2-Expressing Glioma Cells

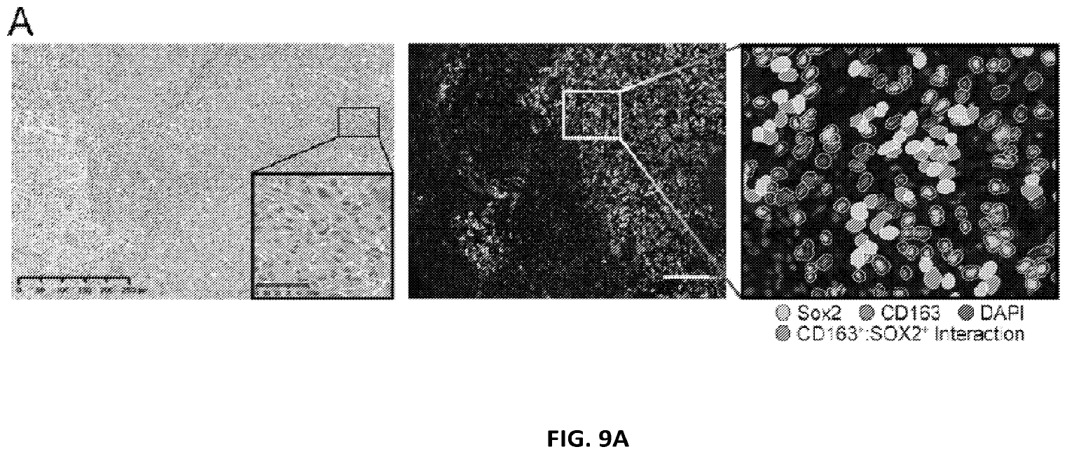

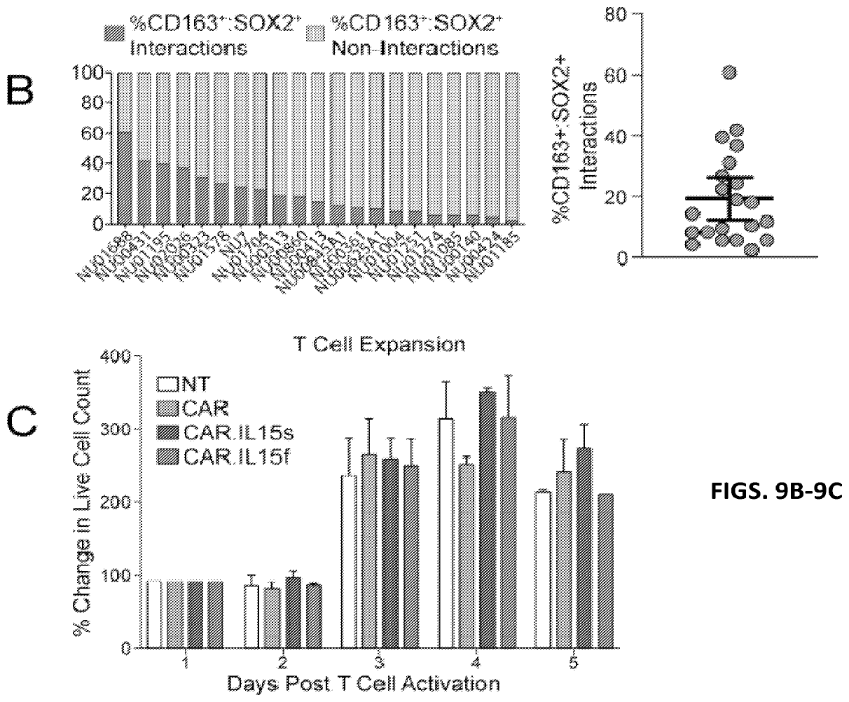

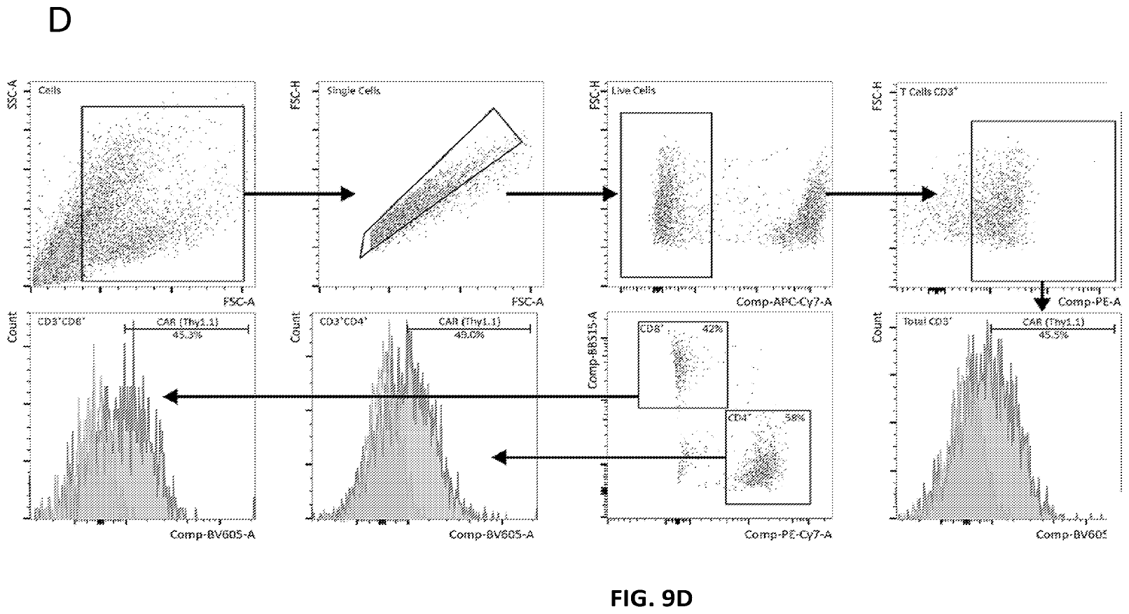

Having confirmed IL15Rα expression in various cells within both human and murine GBM, and demonstrating the capacity of these cells to enhance T cell proliferation, the inventors hypothesized that the addition of IL15 modalities to the IL13Rα2-CAR.CD28.ζ T Cells (CAR T cells)¹⁷ would further potentiate their therapeutic efficacy. Additionally, given that (i) IL15 action requires trans-presentation through IL15Rα expressed predominately on myeloid cells, (ii) the low bioactivity of the single-chain IL15 in biological systems^{34, 35}, and (iii) the multiplex staining of tissue sections from GBM patients (N=25) showing that ˜20% (mean=19.39, 95% CI: 12.39-26.39) of glioma cells (SOX2⁺) are in direct contact with macrophages (CD163⁺) (FIGS. 9A and 9B), the inventors hypothesized that fusing IL15 to the scFv portion of CAR may allow for better bioavailability to direct interactions with both IL13Rα2-glioma cells and IL15Rα-expressing MDSCs (FIG. 3B). To test both hypotheses, the inventors generated the following retroviral vectors for subsequent T cell transduction as previously described¹⁷: (i) a control vector encoding a non-functional CAR (CAR.Δ), (ii) a functional CAR, (iii) a CAR encoding cDNA with a secretable IL15 (CAR.IL15s) (using a 2A self-cleaving peptide), and (iv) a CAR cDNA, where IL15 fused to the N-terminal part of the heavy chain of the scFv IL13Rα2 (clone 47) via (Gly₄S)₃ flexible linker (CAR.IL15f) (FIG. 3A). The AlphaFold2 modeling of scFv47, an antigen-binding portion of CAR T cells, versus IL15 fusion with the scFv47 antibody, shows that the IL15-scFv fusion could be modeled with a high confidence (FIG. 3C). The transduction efficiency of the CD4+ and CD8+ T cells was similar across of all constructs (FIG. 3D), and there was no difference in viability and CAR T cell expansion (FIG. 3E, FIG. 9C). Following transduction, flow cytometry analysis showed no difference in surface expression of IL15 between non-transduced (NT), conventional CAR (CAR), or CAR.IL15s T cells, but a significant increase in surface IL15 expression was seen in CAR.IL15f T cells (FIG. 3F). Expression of T cell memory markers was also significantly higher in CAR.IL15f T cells compared to NT, CAR, and CAR.IL15s T cells (FIG. 3F). To test the functionality of all constructs, the inventors co-cultured NT, CAR, CAR.IL15s, and CAR.IL15f T cells with either parental or IL13RA2 expressing CT2A and GL261 glioma cells. All three constructs displayed cytotoxic capacity against glioma cells in an antigen-dependent fashion, with the highest activity seen in CAR. IL15f T cells (FIG. 3G).

CAR.IL15f T Cells Mediate the Killing of MDSC Cells and Modulate their Immunosuppressive Phenotype

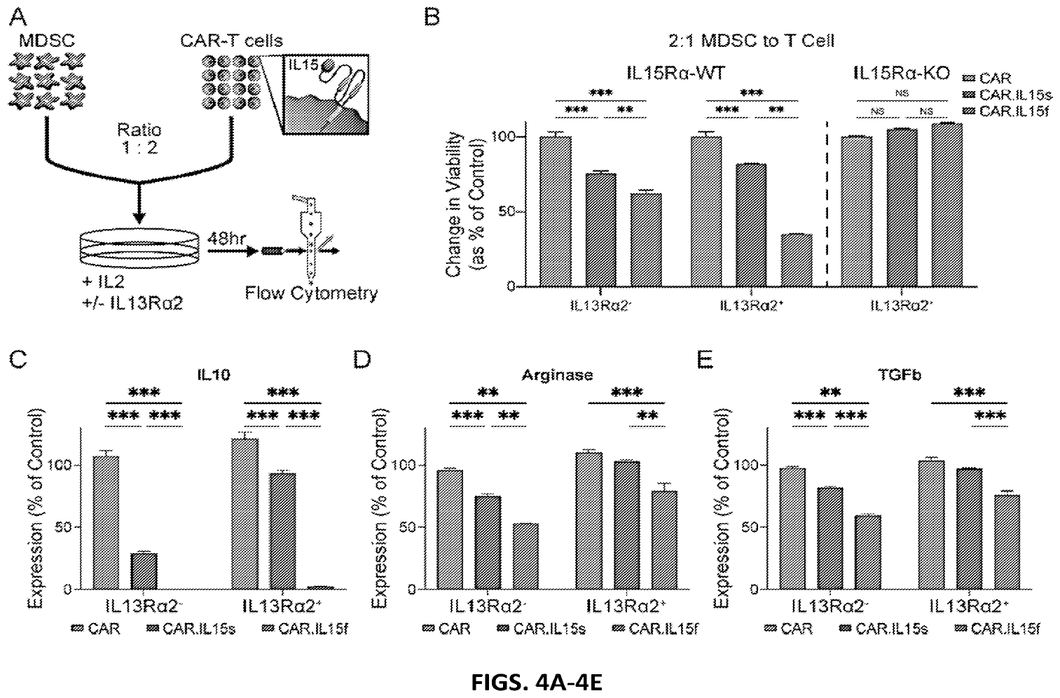

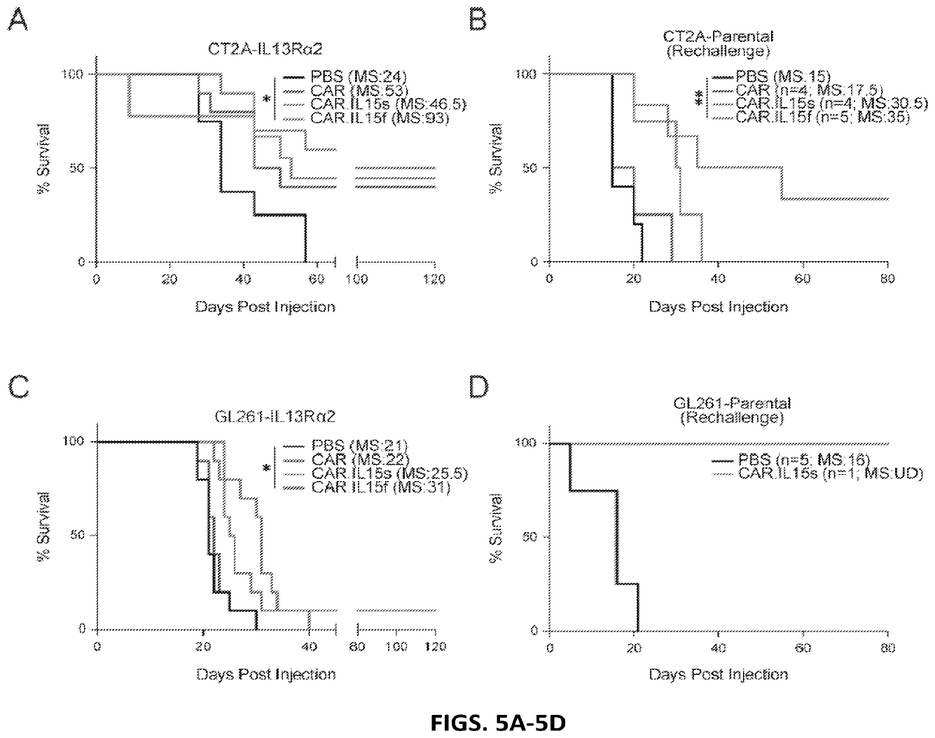

To directly compare the functional activity of CAR.IL15s and CAR.IL15f against MDSCs, CAR T cells were co-cultured at a 2:1 ratio of CAR T cells to MDSCs for 48 hr (FIG. 4A). Flow cytometry analysis showed a greater loss in the viability of MDSCs co-cultured with CAR.IL15f in comparison to CAR.IL15s and conventional CAR T cells. This effect was fully abrogated in co-cultures with MDSCs derived from IL15Rα KO mice (FIG. 4B). Additionally, decreased viability of MDSC was more pronounced when CAR T cells were stimulated with IL13Rα2. The analysis of MDSC for the production of II. 10, Arginase 1 (Arg1), and TGF-β revealed significant decreased levels of these immunosuppressive molecules in co-culture with both CAR.IL15s and CAR.IL15f (FIGS. 4C-4E). In a similar fashion, this effect was also more pronounced when CAR T cells were activated with IL13Rα2. Thus, the data show that IL15-modified CAR T cells exert a direct cytotoxic effect on MDSCs and modulate the immunosuppressive function of MDSCs mediated by IL15/IL15Rα interaction with CAR.IL15f, and that CAR.IL15f is superior to CAR.IL15s in these actions.

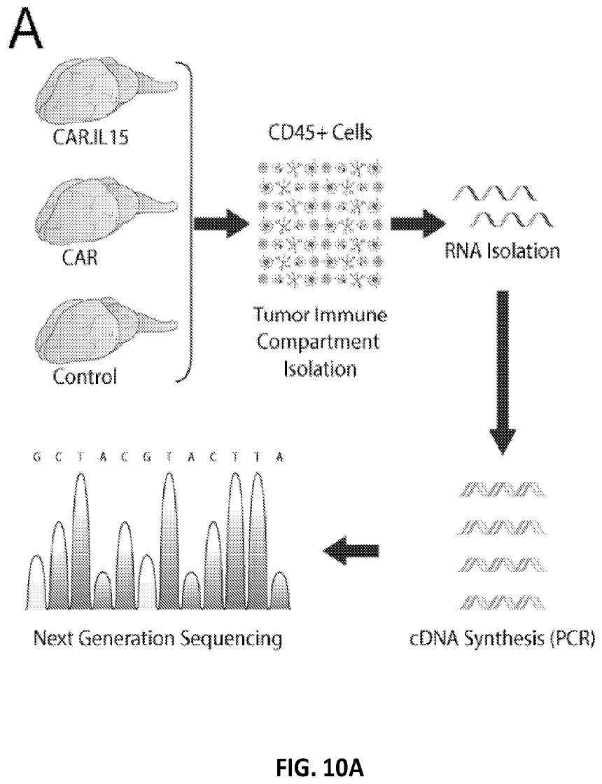

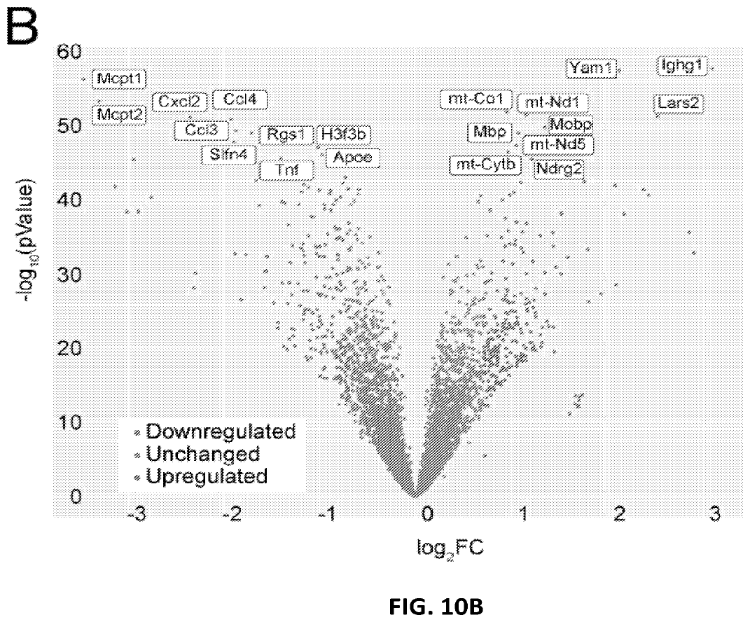

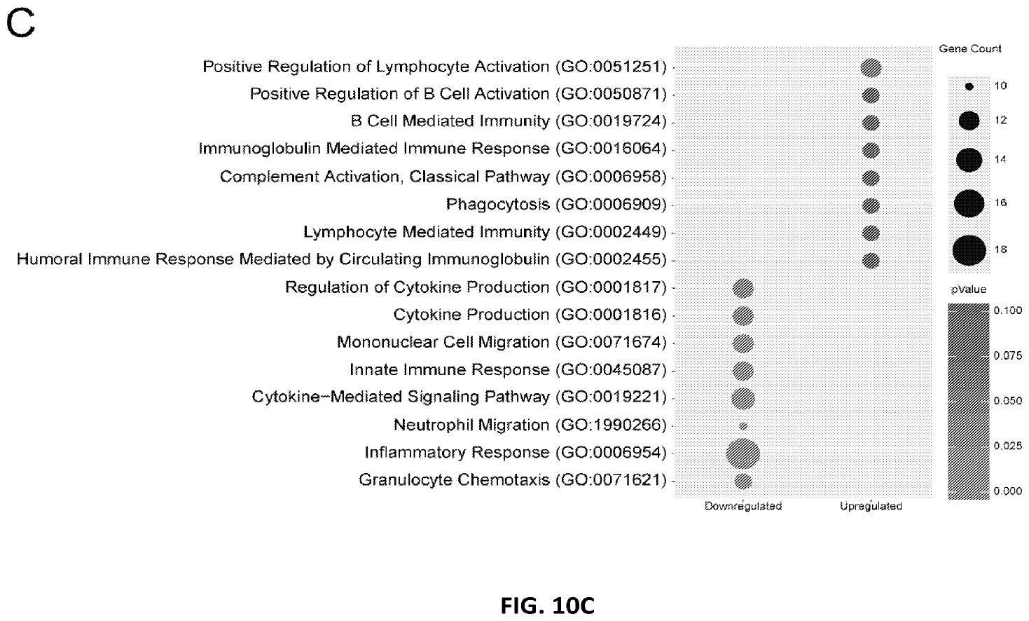

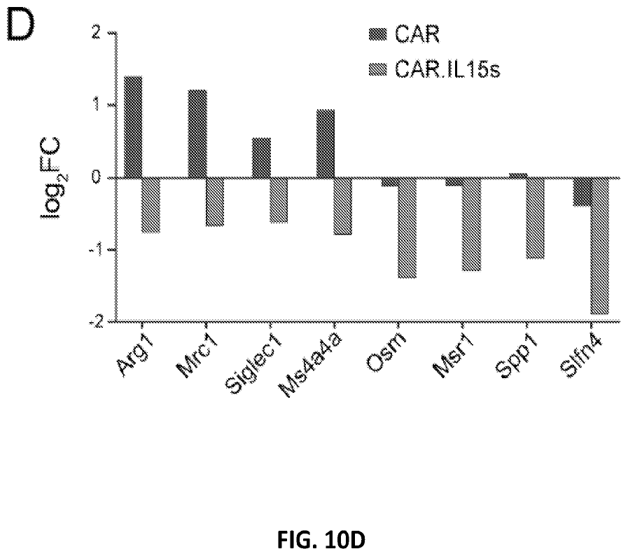

The ability of CAR T cells modified with IL15 to modulate the TME was further confirmed by bulk RNAseq analysis of immune cells (CD45⁺) harvested from GL261 tumors of mice treated CAR T cells (PRJNA908873; FIG. 10A). Violin plot shows the top differentially expressed genes (DEG) between CAR.IL15s compared to conventional CAR T cells and PBS (FIG. 10B). Gene ontology (GO) pathways analysis of DEGs revealed the most significant changes are related to chemokine-mediated signaling pathways, response to chemokines, and leukocyte migration (FIG. 10C). Consistent with in vitro data, the downregulation of genes associated with an immunosuppressive function of tumor-associated macrophages was observed in tumors from mice treated with CAR.IL15s (FIG. 10D). Collectively, these data shows that IL15-armored CAR T cells have the ability to either modulate the immunosuppressive function of MDSCs or deplete them from the TME.

### IL15-Modified CAR T Cells Improve the Survival of Mice in Two Syngeneic Murine Models of GBM

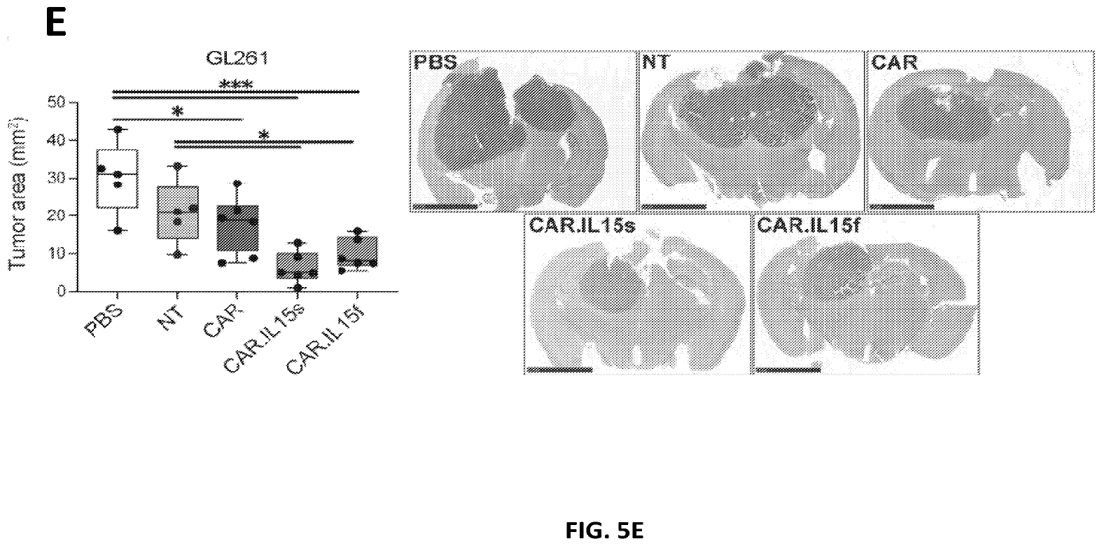

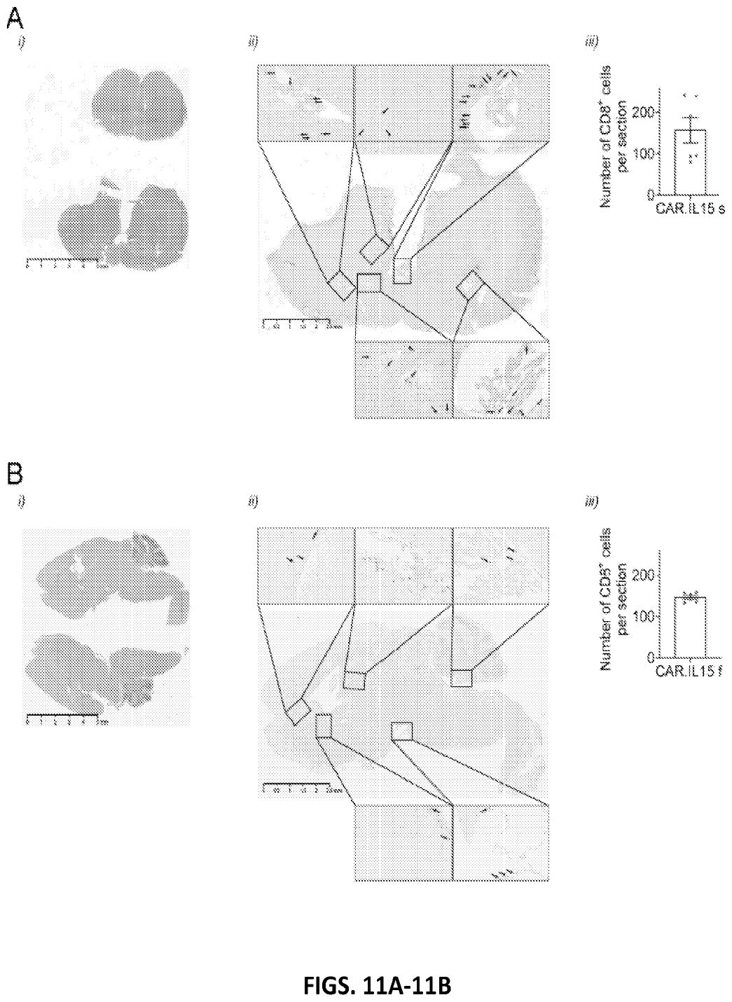

The inventors next investigated the therapeutic properties of IL15-modified CAR T cells in comparison to conventional CAR T cells in vivo using syngeneic murine models of GBM. Mice bearing CT2A or GL261 orthotopic tumors expressing IL13Rα2 were treated via direct intra-tumoral injection (i.t.) with 1×10⁶ CAR T cells. In the CT2A model, mice treated with CAR.IL15f had a superior median survival of 93 days in comparison to 46.5 days in CAR.IL15s, 53 days in CAR, and 34 days in PBS group (n=10, *p<0.05) (FIG. 5A). Long-term surviving mice from each group were re-challenged with parental CT2A cells; age-matched mice served as the control. Upon the re-challenge, the mice from the CAR. IL15f group showed superior survival over mice treated with CAR.IL15s T cells or unmodified CAR T cells groups (FIG. 5B). Histological analysis of brain tissue confirmed a complete tumor regression, with CD8⁺ T cells present at the site of injection and in other areas of the brain (FIG. 11A). In GL261 models, the treatment of IL15-modified CAR T cells resulted in improved albeit modest survival in GL261 model, with better survival seen in mice treated with CAR.IL15f compared to all other groups (FIG. 5C). The long-term surviving animal was also resistant to re-challenge with parental GL261 line (FIG. 5D). Histological analysis of tissue showed no tumor in the brain of the re-challenged animal (FIG. 11B). To better understand the survival analysis, the inventors measured the area of tumors in all experimental groups of the GL261 model (FIGS. 5E-5F). Mice treated with IL15-modified CAR T cells had 2-3 times smaller tumors than tumors in CAR or non-treated control groups. Collectively, these experiments show the IL15-modified CAR T cells provide a survival benefit for tumor-bearing animals and further confirm that IL15-modification enhances the functionality and therapeutic efficacy of CAR T cells. Histopathologic examination of the tissues showed no evidence of focal or diffuse meningoencephalitis, demyelination, neuronal dropout, infarction, or vasculitis.

### IL15-Modified CAR T Cells Modulate the TME by Diminishing the Myeloid Compartment and Increasing Frequencies of the Host's CD8⁺, NK, and B Cells

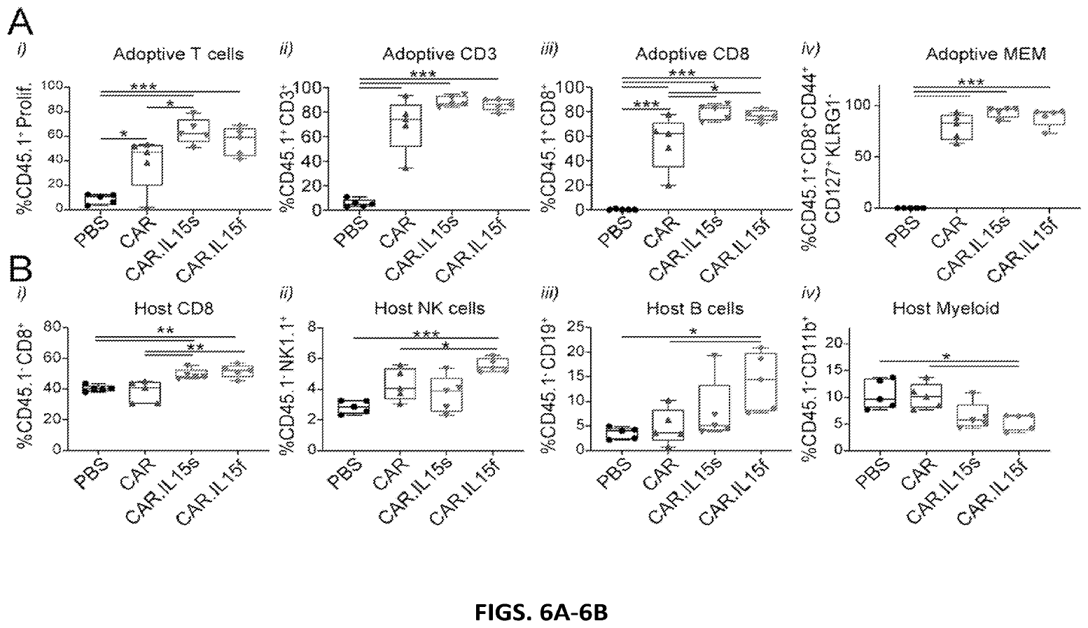

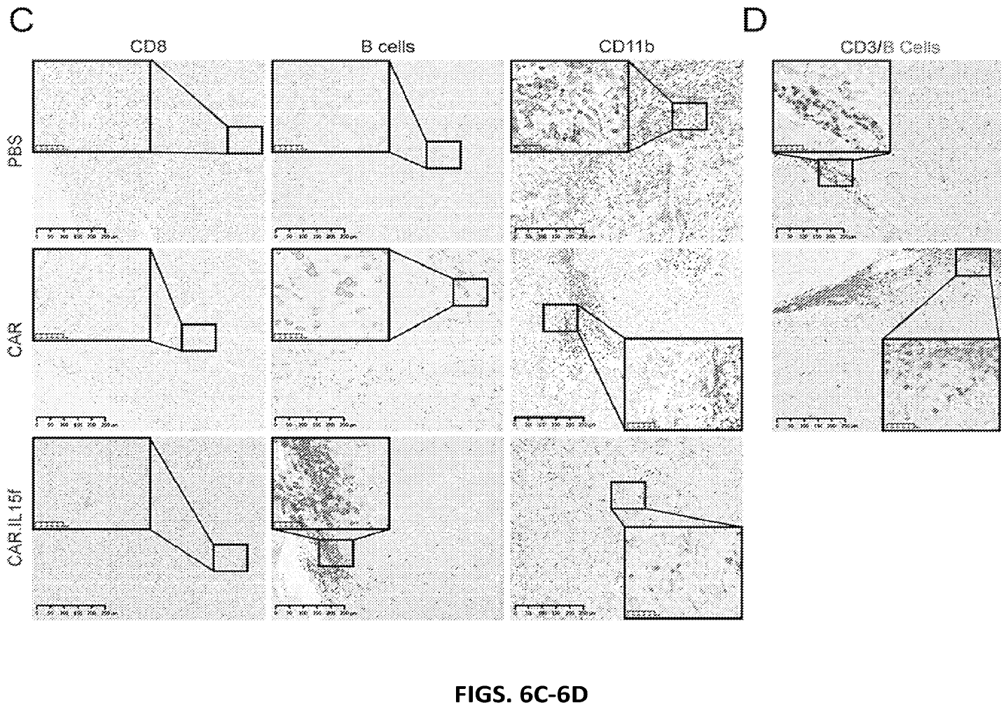

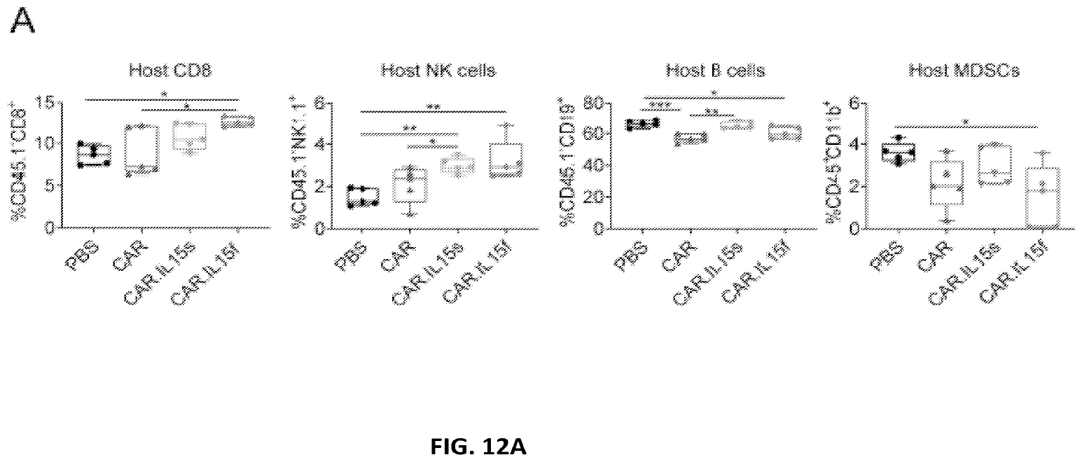

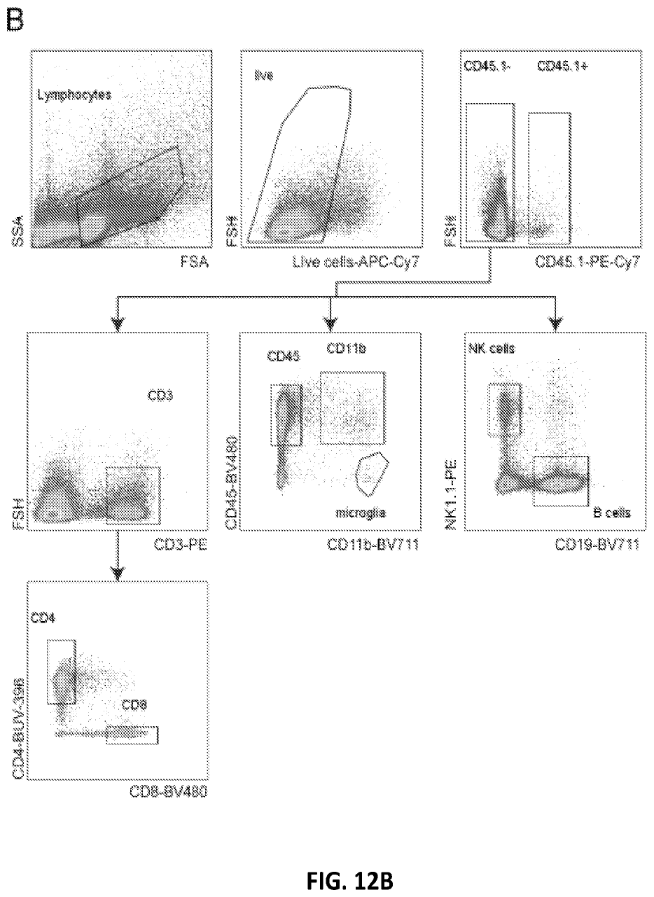

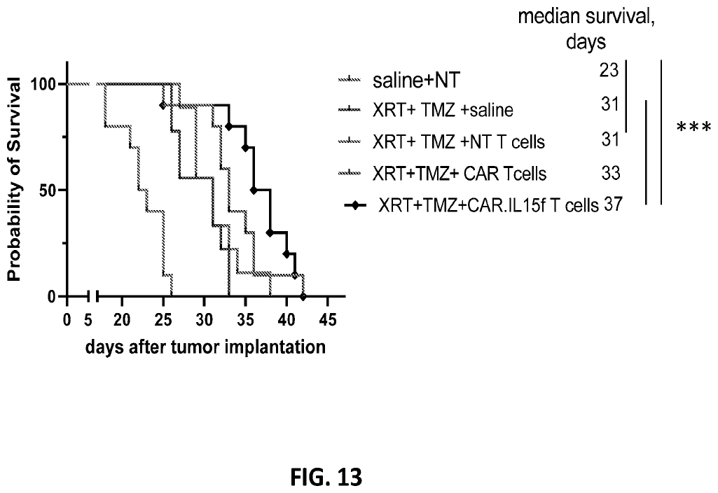

To gain further insights into the therapeutic properties of IL15-modified CAR T cells, the inventors treated GL261-bearing mice with CAR T cells, IL15-modified CAR T cells, or PBS, and analyzed the TIME of animals 7 days post treatment. To distinguish between host and adoptive T cells, the inventors used CD45.1 congenital mice to generate CAR T cells and adoptively transferred them into CD45.2 mice. The data showed (i) increased adoptive T cell proliferation, and higher frequency of (ii) CD3⁺, (iii) CD8⁺ in the TME of mice treated with CAR.IL15s and CAR.IL15f T cells compared to CAR T cell and PBS groups (FIG. 6A). No difference in (iv) memory phenotype of adoptively transferred cells was detected between groups (FIG. 6A). Analyzing the host cells of the TME, the inventors observed increased (i) CD8⁺ T cells, (ii) NK, and (ii) B cells seven days post CAR T cells treatments compared to PBS, with CAR.IL15f T cell group being superior to the other CAR T cell group (FIG. 6B). The inventors also observed a two-fold decrease in the frequency of (iv) myeloid cells (CD45⁺ and CD11b⁺) when treated with CAR.IL15s and CAR.IL15f (FIG. 6B). Histological analysis of tissues revealed CD8⁺ T cells were distributed throughout the tumor, and there was an increased presence of B cells within tumors treated with CAR.IL.15f (FIG. 6C). Moreover, dual staining of tumors showed colocalization of B and CD3⁺T cells within the brains of CAR.IL15f treated mice (FIG. 6D). Staining for CD11b showed lower intensity within tumors treated with CAR.IL15f compared to all other groups (FIG. 6C). To assess the distribution in extracranial sites, the inventors assessed the immune compartment in spleens of mice treated in all groups. Small changes in the frequency of CD8⁺ T cells, NK, B, and CD11b⁺ cells were observed in the spleen (FIG. 12), suggesting that the local treatment with CAR T cells, to a lesser degree, may also influence peripheral immune compartments.

CAR.IL15f T Cells Improve Survival of Mice Bearing CT2a-IL13Rα2 Tumors Treated with Whole Brain Irradiation (XRT) and Temozolomide Therapy (TMZ)

The inventors examined the effect of CAR.IL15f T cells on survival of mice implanted with CT2A-IL13Rα2 tumors and treated with irradiation or chemotherapy. Whole brain irradiation (XRT) and temozolomide therapy (TMZ) treatment significantly improved the survival of mice in comparison to control (saline+NT cells). Treatment of mice with CAR.IL15f T cells further improved the survival of mice in comparison to XRT+TMZ groups. These data suggest that CAR.IL15f T cells could be used in combination with a standard of care for GBM, and delivered not only locally in the brain (e.g. intratumorally, or intraventricularly in CSF), but also via systemic intravenous route.

### Discussion

Here the inventors describe that MDSC in the TME of human and murine GBMs express IL15Rα. To target glioma cells and MDSC the inventors generated murine T cells expressing IL13Rα2-CARs and secretory IL15 (CAR.IL15s) or IL13Rα2-CARs in which IL15 was fused to the CAR to serve as an IL15Ra-targeting moiety (CAR.IL15f). CAR.IL15s includes full length IL15, and is secretable due to the 2A domain. The IL15 also includes a signal peptide that assists with its trafficking outside of cells. The inventors demonstrate that CAR.IL15s and CAR.IL15f T cells (i) deplete MDSCs and (ii) decrease their secretion of immunosuppressive molecules in vitro with CAR.IL15f T cells being more efficacious. In vivo, CAR.IL15f T cells reversed the immunosuppressive TME to a greater extent than CAR or CAR.IL15s T cells, resulting in improved survival in two glioma models.

The inventors have demonstrated that transgenic expression of IL15 improves the effector function of CAR T cells against solid tumors, including glioma.^{15, 24, 36} However, it was unclear if IL15Rα is expressed on myeloid cells, which comprise up to 30-50% of the GBM tumor mass³⁷, and can be exploited as an immunotherapeutic target. Taking advantage of the TCGA data set the inventors demonstrated that IL15Rα is expressed in glioma and that expression correlates with histological grade. IL15Rα expression colocalized with CD163 expression, a marker of macrophages, as judged by IHC analyses. Further, analyses of paired peripheral blood and tumor-infiltrating cells of GBM patient samples revealed significantly higher IL15Rα expression in immune cells of brain tumors compared to those of peripheral blood. Since the myeloid compartment of GBMs consists predominantly of MDSCs, this data suggested that IL15Rα is expressed by MDSCs. IL15Rα expression by MDSCs was confirmed in two (GL261, CT2A) murine glioma models. In addition, higher IL15Rα expression in myeloid cells of the TME compared to the peripheral compartment was also observed in murine models, mirroring the findings in humans. To the inventors' knowledge, this is the first study that demonstrates preferential expression of IL15Rα in myeloid cells or the GBM TME, providing the impetus to conduct mechanistic studies in the future.

To explore if IL15Rα expression could be leveraged therapeutically to enhance the anti-glioma activity of IL13Rα2-CAR T cells the inventors generated two CAR T cell effector population. One expressing secretory IL15 (CAR.IL15s) and one in which IL15 was directly fused to the IL13Rα2-CAR (CAR.IL15f T cells). Of interest, the inventors did not observe fractercide of CAR.IL15f T cells. Studies have indicated that spacer length between the ligand and cell membrane modulates fractercides, with longer spacers being protective.³⁸ Since in the CAR.IL15f design, the scFv of the CAR is between the IL15 molecule and the cell membrane, creating a significant distance, which likely protects from fratricide.

The inventors compared the ability of CAR, CAR.IL.15s, and CAR.IL15f T cells to reverse the immunosuppressive effects of MDSCs in coculture assays in vitro. While both IL15-modified CAR T cell populations were able to decrease MDSC-mediated immunosuppression as judged by downregulating the expression of IL10, Arginase-1, and TGF-β, CAR.IL15f T cells were more effective in comparison to CAR.IL15s T cells. Indeed, CAR.IL15f T cells also reduced MDSC viability, most likely through direct cytotoxic effects. In vivo, both IL15-modified CAR T cell populations extended the survival of animals bearing GL261 and CT2A gliomas, again with CAR.IL15f T cells being more efficacious. Neither CAR.IL15s or CAR.IL15f T cells induced toxicities post intratumoral injection in the glioma models.

To better understand the differences in survival benefits between the CAR T cell populations, the inventors characterized the immune compartment within the TME post therapy. In agreement with the in vitro studies, the inventors observed a reduction of MDSC frequency in mice treated with IL15-modified CAR T cells, with CAR.IL15f T cells having greater anti-MDSC activity, further confirming the dual targeting ability of the designed CAR T cells. Bayik and colleagues have recently demonstrated that different subsets of MDSCs can drive immunosuppression in glioma tumors in a sex dependent manner.³⁸ Whether response of different subsets of MDSCs to CAR T cells therapy depends on an animal's sex will have to be determined in a future studies. B and NK cells, are normally found at low frequencies within the GBM TME.³⁹ The presence of tertiary-like lymphoid structures and activated B cells within tumors has previously been established as positive predictors of patient survival in several types of cancer^{40,41, 42}, and recruitment of NK cells has been reported to correlate with a better prognosis in gliomas.^{43, 44} Although tertiary-like lymphoid structures in the brains of mice were not fully characterized, the inventors observed a colocalization of B and CD3⁺ T cells, and an increase in B and NK cells frequency following IL15-modified CAR T cell therapy. Given the increased survival post rechallenge with parental glioma cell lines that do not express IL13Rα2, it is possible that IL15-modified CAR T cells aid in the induction of endogenous immune responses to promote antigen spreading. Other investigators have explored whether targeting TAMs with CAR T cells redirected to CD123 or FRβ improves CAR T cell efficacy in preclinical lymphoma or ovarian cancer models.^{45, 46} Only one of these studies was conducted in an immune-competent animal model, timing was critical, and FRβ-CAR T cells only improved the antitumor activity of tumor-specific CAR T cells if there were given prior to their infusion. In their approach, the inventors targeted tumor cells and MDSCs simultaneously, and based on the aforementioned study, exploring a multiple-dosing regimen might be advisable in the future.

In conclusion, the study shows that modifying CAR T cells with IL15 improves their therapeutic properties. Furthermore, the inventors show that the fusion of IL15 to the antibody part of CAR T cells generates a dual targeting system that not only diminishes the frequency of MDSC and glioma cells, but also modulates their immunosuppressive phenotype. Most importantly, the studies open the opportunity for investigating other targeting moieties on the surface of MDSCs, specifically those enriched in cells of GBM TME, and applying these modifications to CAR T cells for their direct dual functions against glioma cells and immunosuppressive MDSC.

### Materials and Methods

### Cell Culture

Phoenix-eco cells were originally obtained from Thermo Fisher Scientific. Murine glioma cell line GL261 was obtained from NIH. CT2A cells were obtained from Dr. T. Seyfried (Boston College, Chestnut Hill, MA). GL261-hIL13Rα2 and CT2A-hIL hIL13Rα2 were modified as previously described¹⁷.All glioma lines were grown in Dulbecco's Modified Eagle Medium (DMEM; Invitrogen, Grand Island, NY) supplemented with 10% fetal bovine serum (FBS). Isolated murine T cells were maintained in complete RPMI 1640 (Invitrogen, Grand Island, NY). Mycoplasma screening was performed using MycoAlert™ Mycoplasma Detection Kit (Lonza, Walkersville, MD) every 3-6 months.

### Generation of Retroviral Constructs and CAR T Cells.

The IL13Rα2-CAR.CD28.ζ and IL13Rα2-CAR.Δ were cloned in the pRV2011 (M) vector, as previously described¹⁷. For the generation of the IL15 constructs, we (i) subcloned a cDNA encoding T2A and murine IL15 at the C-terminal part of the CAR construct to generate a retroviral vector that encoded the CAR, T2A, and secretory IL15 (IL15s), or (ii) subcloned murine IL15 and (Gly4S)3 linker at the N-terminus part of CAR to generate an IL15-CAR fusion protein (IL15f). All CAR constructs were verified by Sanger′ sequencing (GenScript, Piscataway, NJ).

To generate retroviral particles, Phoenix-eco cells were transfected with the CAR construct using GeneJuice (Sigma-Aldrich, St. Louis, MO). 48 and 72 hr post-transfection, supernatants containing retrovirus were collected and subsequently used to generate CAR T cells, as previously described.¹ CD3⁺ T cells were isolated from the spleens of mice using a Mouse T Cell Isolation Kit (STEMCELLS Technologies, Cambridge, MA). The activated T cell were transduced with replication-deficient retrovirus encoding for CARs for two consecutive days starting 24 hrs post T cell stimulation with anti-CD3 and anti-CD28 monoclonal antibodies (Biologend, San Diego, CA). Cells were allowed to rest for 2 days in RMPI 1640 media supplemented with 10% FBS and 50 ng/ml of IL2 (Peprotech, Cranbury, NJ).

### Modeling IL15-scFvIL13Rα2 Fusion in AlphaFold

The sequences of the single-chain variable fragment (scFv) IL13Rα2 (scFvIL13Rα2) molecule^{27,28} and the murine IL15 (gene bank accession number NM_008357.1) fusion molecule were uploaded to the Alphafold2 software.²⁹ With the software, the inventors constructed multiple sequence alignments (MSAs), and then generated ten possible predictions of each molecule's structure. For each molecule, the inventors generated five predictions that used Alphafold's Amber force field procedure, a procedure that removes stereochemical aberrancies but that does not improve a prediction's accuracy (as measured by its pLDDT-Cα score), and five other predictions without procedure. The inventors visualized the best predictions of each of IL-15+scFv antibody's and scFv antibody-alone's structures in Google Colab (as measured by pLDDT-Ca), using the py3Dmol, matplotlib.pyplot, and ipywidgets packages in Python and a Python3 kernel. The color of the molecules at any given point reflects Alphafold's confidence in its prediction for the three-dimensional structure at that point. Alphafold measures the distance in a three-dimensional space between where its prediction estimates it is and where an actual molecule's amino acid would be, which was a measurement reflected in the colors of the molecule. The measurements are predicted Local Difference Distance Test scores (pLDDT-Cα scores). LDDT-Cα scores take into account all atoms of a prediction, including side-chain atoms, and are therefore able to accurately score a model's prediction of e.g., the local geometry in a binding site. LDDT-Cα scores measure how well the predicted structure of a molecule would match the experimentally determined structure of that molecule; it does this by assembling homologous amino acid sequences and then gauging how consistently those sequences fold, and then the model uses this analysis to calculate how likely it is to predict this sequence's folding well. This calculation is the pLDDT. The inventors used Alphafold 2.0.0 and Python 3.7; they set the “preset” parameter to “casp14” so that the model would use the same settings as it used in the protein-prediction competition CASP 14, and they set the “max_template_date” to Sep. 10, 2021.

### Flow Cytometry

Flow cytometric analysis was performed using the BD FACSymphony A5-Laser Analyzer (Becton Dickinson, Franklin Lakes, NJ) at the Robert H. Lurie Comprehensive Cancer Center Flow Cytometry Core Facility. Cells isolates were blocked with PBS supplemented with 2% FBS and mouse Fc blocking reagent (1:200) (CD16/32, BioLegend, San Diego, CA). Surface proteins were stained through incubation of cells with fluorochrome-conjugated antibodies (BioLegend, San Diego, CA, unless specified) on ice for 20 minutes. To stain intracellular proteins, cells were fixed and permeabilized (Invitrogen, Waltham, MA, #00-5523-00) before incubation with antibodies. eBioscience Fixable Viability Dye eFluor 780 (1:1000 in PBS) (Invitrogen, Waltham, MA) was used to distinguish viable cells. Lastly, labeled cells were washed with PBS before being analyzed. All antibodies used in the study are listed in Table 1.

TABLE 1

| Marker | Fluorophore | Company | Rer/Cat | Clone |
|---|---|---|---|---|
| 41BBL | PE | Biolegend | 107105 | TKS-1 |
| Arginase-1 | APC | Invitrogen | 17-3697-82 | A1exF5 |
| CD103 | BV711 | Biolegend | 121435 | 2E7 |
| CD11b | BV711 | Biolegend | 101242 | N418 |
| CD11c | FITC | Invitrogen | 11-0114-81 | N418 |
| CD11c | APC | Biolegend | 117310 | |
| CD127-biotin | Streptavidin | Invitrogen | 46431780 | |
| | PerCP-eFluor | eBioscience | | |
| | 710 Conjugate | | | |
| CD14 | BV605 | Biolegend | 367125 | 63D3 |
| CD15 | PE | Biolegend | 323005 | WBD3 |
| CD16 | PE-TR | Thermofisher | MHCD1617 | 3G8 |
| CD19 | BV711 | Biolegend | 11555 | 6D5 |
| CD215 (IL-15Rα) | PE | Biolegend | 153503 | 6B4C88 |
| CD3 | PE | Biolegend | 100206 | 17AZ |
| CD3 | AF700 | Biolegend | 100215 | 17AZ |
| CD33 | PE-Cy7 | Biolegend | 303433 | WM53 |
| CD4 | BUV395 | BDbiosciences | 740208 | RM4.5 |
| CD44 | PerCP/Cyanine0.5 | Biolegend | 103032 | IM7 |
| CD44 | PE | Biolegend | 103008 | IM7 |
| CD45 | BV510 | Biolegend | 103138 | SB/199 |
| CD45 | PECy7 | Biolegend | 110730 | A20 |
| CD45.1 | PECy7 | Biolegend | 110730 | A20 |
| CD62L | PerCP | Biolegend | 104429 | MEL14 |
| CD8 | FITC | Biolegend | 100723 | 53-6.7 |
| CD8 | BV605 | Biolegend | 100744 | 53-6.7 |
| CD8 | AF700 | Invitrogen | 56-0081-82 | 53-6.7 |
| CD8 | BV510 | Biolegend | 100751 | 53-6.7 |
| CellTrace ™ Violet | CTV | Invitrogen | C34571 | |
| Fixable Viability Dye | eFluor ™ 780 | Invitrogen | 65-0885-14 | |
| Gr8 | APC | Biolegend | 322204 | QA16A02 |
| IFNg | AF700 | ebioscience | 46431780 | XMG1.2 |
| IL15Ra | APC | Biolegend | 330209 | IM7A4 |
| KLRG1 | APC | Biolegend | 138411 | ZF1/KLRG1 |
| Ly6C | AF700 | BD Biosciences | 561237 | AL-21 |
| Ly6G | PerCP-Cy5.5 | Biolegend | 127615 | 1AB |
| NK1.1 | PECy7 | Biolegend | 108713 | PK136 |
| Th1.1 | BV605 | Biolegend | 202537 | Ox-7 |
| XCR-1 | PerCPi | Biolegend | 148207 | ZET |

### Functional Analysis of CAR T Cells

The cytotoxicity of all CAR T cells against IL13Rα2-expressing glioma cells was determined in a co-culture assay with increasing ratios of the effector (CAR T cells) to target (glioma) cells as the inventors previously described.¹⁷ Briefly, parental GL261 or CT2A, or IL13Rα2-expressing GL261-IL13Rα2 and CT2A-IL13Rα2 glioma cells were labeled with CellTrace™ CSFE dye (Invitrogen, Waltham, MA) prior co-culture with CAR T cells. After 48-72h, cells were collected and stained with an eBioscience Fixable Viability Dye eFluor 780 (1:1000 in PBS) (Invitrogen, Waltham, MA) and anti-CD45 antibodies. A fraction of live CD45-negative (e.g., glioma) cells was assessed by flow cytometry.

### Processing of Patients' Blood and Tumor Samples

The study was approved by the Institutional Review Board (IRB #STU00095863). Prior the use in the study, the patient's tumor and blood samples (PBMCs) were de-identified. Patient consents were obtained prior to biospecimen collection. Isolation of T cells from PBMC and tumor tissue was performed as previously described.¹⁷

Generation of Murine MDSC from Bone Marrow for In Vitro Assays

Bone marrow (BM) cells from C57BL/6 mice were flushed out from femurs with complete RPMI using a 10 mL syringe and 25-gauge needle into complete RPMI (Corning). BM cells were centrifuged (10 minutes, 400 g, at 4° C.), and red blood cells were lysed using ACK lysing buffer (Sigma) for 5 minutes at RT. Cells were washed with PBS, pelleted, resuspended in RPMI, counted, and plated into 24-well plates (Corning) at a density of 2.5×10⁵ cells per well in 50% complete RPMI and 50% conditioned media supernatant that was collected from CT2A cells (plated at 2×10⁶ CT2A cells for 72 hours), supplemented with GM-CSF (40 ng/ml) (PeproTech, Rocky Hill, NJ) and IL-6 (40 ng/ml) (PeproTech, Rocky Hill, NJ). After 3 days of culture, the media was removed, and replaced with fresh media. Six days after BM isolation, cells were collected, washed with PBS, and used in the functional assays described below. This protocol was adopted from a previously established method.^{5, 6}

### Co-Culture of MDSCs and CAR T Cells

To assess the effect of CAR T cells on MDSC, CAR T cells were activated with recombinant IL13Rα2-covered plates for 16-18 hours. MDSC and CAR T cells were then combined at various ratios. After 48-72 hours, cells were collected and stained with eBioscience Fixable Viability Dye eFluor 780 (Invitrogen, Waltham, MA). MDSC also were stained for intracellular IL-10, TGFβ, and Arg-1 (Biolegend, San Diego, CA)

### RNA-Seq and Data Analysis

For RNAseq analysis of expression of IL15Rα in Gr1⁺ cells, CT2A tumors and spleens were harvested from 10 mice in each of 3 independent experiments two weeks after tumor implantation. Brains and spleens were processed into a single-cell suspension, and Gr1⁺ cells were isolated using biotinylated anti-Gr1 antibodies and streptavidin-conjugated magnetic beads (Miltenyi Biotech, Auburn, CA). Gr1⁺ cells total RNA was extracted using TRIzol (Thermo Fisher Scientific) reagent. For RNAseq analysis of mice bearing G1261-IL13Rα2 tumors treated with either PBS or CAR T cells (n=3/group), tumors were collected seven days after CAR T cells treatment and processed into a single-cell suspension for subsequent isolation of CD45⁺ cells using anti-CD45 antibodies and streptavidin-conjugated magnetic beads (Miltenyi Biotech). Chloroform (200 μl) was added to TRIzol samples to separate liquid phases, and RNA was precipitated from the aqueous phase with 70% isopropanol. RNA was dried, reconstituted in RNase-free water, and sequenced (Novogene, Durham, NC). All data were presented as total read counts and fragments per kilobase per million (fpkm) reads.

### Quantitative Real-Time Polymerase Chain Reaction (RT-qPCR)

Total RNA was isolated from murine Gr1⁺ cells purified from the brain and spleens of tumor-bearing mice using an RNeasy plus kit (Qiagen, Boston, MA). About 500 ng RNA was reverse-transcribed using an iScript cDNA conversion kit (Bio-Rad, Hercules, CA). RT-qPCR was carried out using SYBR green kit (Bio-Rad, Hercules, CA, USA) using primers for human and murine IL15Rα (Human F: 5′-GTGGCCCTGTGGATACACAC-3′ (SEQ ID NO: 10), Human R: 5′-ACAACAGCAGCTATTGTCCCG-3′, (SEQ ID NO: 11), Murine F: 5′-AATCAGATACCGCAATGACCAC-3′, (SEQ ID NO: 12), Murine R: 5′-CAGAAGTTGTTTGGGATGGTGT-3′, (SEQ ID NO: 13)) and pair of primers for ß-Actin (R: 5′-ACATCTGCTGGAAGGTGGAC-3′, (SEQ ID NO: 14), F: 5′-TTGCTGACAGGATGCAGAAG-3′, (SEQ ID NO: 15)). The 2-ΔΔCT method was used for the analysis of the relative expression of IL15Ra, and all sample values were normalized to the β-Actin expression.

### Multiplex Immunofluorescence for Visualization of the Interaction Between Macrophages and GBM Cells

De-identified human tissue sections were obtained from the Nervous System Tumor Bank of the Northwestern University after IRB approval (STU00206457-CR0001). Five μm sections were obtained from formalin-fixed paraffin-embedded (FFPE) human GBM samples. In brief, deparaffinization of slides was performed using xylene followed by dehydration with ethanol. Next, these slides were fixed with 3% hydrogen peroxide in methanol and were subjected to antigen retrieval using pH6 citrate buffer or pH9 EDTA buffer. CD163 (#ab213612, clone EPR19518, Abcam, dilution 1:600) and SOX2 (#ab92494, clone EPR3131, Abcam, dilution 1:5000) were the markers employed to identify macrophages and tumor cells, respectively. Visualization was done using tyramide signal amplification with the Opal 7-color IHC Kit (#NEL821001KT, Akoya Biosciences, Menlo Park, CA). Opal 570 (dilution 1:800) was paired with CD163, and Opal 620 (dilution 1:150) was paired with SOX2. Staining for these markers was performed with cyclic steps involving antigen retrieval step, protein blocking, epitope labeling, and amplification of the signal. Spectral DAPI (Akoya Biosciences) was used to counterstain slides, followed by the application of a long-lasting aqueous-based mounting medium.

Multispectral imaging (MSI) was performed using the Vectra 3 Automated Quantitative Pathology Imaging System (Akoya Biosciences). To acquire spectral images, whole slide scans were performed to select tumoral regions. Next, MSI was acquired to capture fluorescent signals and generate files for further processing. A spectral library was created for all fluorophores, which was used to subject images to spectral unmixing in Form Tissue Finder software (inForm 2.4.9, Akoya Biosciences) that allow visualization of the Opal dye signal derived from CD163 and SOX2 markers. After adjusting cell segmentations settings employing DAPI for nuclear segmentation, a machine-learning algorithm within inForm was applied to identify SOX2⁺ and CD163⁺. The processing of images from tumor samples was exported. Unmixed images were processed in R using R packages Phenoptr and PhenoptrReports to merge and create the consolidated files. The spatial map viewer within R allowed visualization cells in contact between SOX2⁺ and CD163⁺.

### Animal Experiments

All animal experiments were performed according to the protocols approved by the Northwestern University Institutional Animal Care and Use Committee, protocol IS00009472. CD45.1, CD45.2 C57BL/6 and IL-15Ra KO mice were obtained from Jackson Laboratory. As previously described, male and female 6-8 weeks old mice were utilized for intracranial glioma implantation¹⁷. In 7 days after tumor implantation, animals received an intratumoral (i.t.) injection of saline, non-transduced (NT) or 1×10⁶ CAR T cells. All animals were then randomly assigned to housing cages, separated by gender, and monitored for survival.

### Immunostaining of Patients and Murine Glioma Tissues

Ten GBM patients' tissue sections were stained to determine the expression of IL15Rα using a polyclonal rabbit antibody (Boster Bio, #A03016-1). Detection of bound antibody was carried out using a donkey anti-rabbit biotinylated antibody (Jackson Immunoresearch, cat #711-065-152) and streptavidin-peroxidase (Jackson ImmunoResearch #016-030-084). For detection of immune cells in murine brains, the inventors utilized antibodies against murine CD11b (Abcam #133357), CD3⁺ (Abcam #16669) and CD8⁺ T cells (Ebioscience #14-0195-82) and B cells (CD45R/B220, BD Biosciences #550286). For dual staining of B and CD3⁺ T cells, the inventors used antibody detection antibodies conjugated with green (Vina Green, Biocare Medical #BRR807a) and red (Warp Red, Biocare medical #WR806) chromogens, respectively.

### Morphometric Analysis of Brain Tumors

To compare the changes in tumor growth between control and CAR T cells treated groups, mice were sacrificed on day 10 after CAR T cells treatment. After perfusion with PBS, brains were collected and fixed in 10% PBS-buffered formalin. The paraffin tissue sections were cut at 4 μm and stained with hematoxylin and eosin (H&E) dye. The inventors then prepared 21 slides from each brain, and every fifth slide was stained for H&E. Stained sections were then scanned and processed in NDP.view 2.8.24 (Hamamatsu Photonics K.K.). Finally, the tumor area was traced to obtain area measurements.

### Statistical Consideration

All statistical analyses of data were performed with GraphPad 9 Software (Prism, La Jolla, CA). As indicated, significance, defined as p less than 0.05 in all statistical tests, was calculated with an unpaired Mann-Whitney test or an unpaired two-tailed Student's t-test. Multiple groups were analyzed with either a one- or two-way ANOVA, followed by a Tukey's multiple comparisons test. Kaplan-Meier plots were generated using GraphPad 8 Prism, and p values for curve comparisons were calculated using the log-rank method. Data are presented as mean±SEM.

### REFERENCES

1. Stupp R, Mason W P, van den Bent M J, et al. Radiotherapy plus concomitant and adjuvant temozolomide for glioblastoma. N Engl J Med. 2005; 352(10): 987-96.
2. Stupp R, Taillibert S, Kanner A, et al. Effect of Tumor-Treating Fields Plus Maintenance Temozolomide vs Maintenance Temozolomide Alone on Survival in Patients With Glioblastoma: A Randomized Clinical Trial. JAMA. 2017; 318(23): 2306-2316.
3. Porter D L, Levine B L, Kalos M, Bagg A June C H. Chimeric antigen receptor-modified T cells in chronic lymphoid leukemia. N Engl J Med. 2011; 365(8): 725-33.
4. Munshi N C, Anderson L D, Jr., Shah N, et al. Idecabtagene Vicleucel in Relapsed and Refractory Multiple Myeloma. N Engl J Med. 2021; 384(8): 705-716.
5. Morgan R A, Yang J C, Kitano M, et al. Case report of a serious adverse event following the administration of T cells transduced with a chimeric antigen receptor recognizing ERBB2. Mol Ther. 2010; 18(4): 843-51.
6. Magee M S, Abraham T S, Baybutt T R, et al. Human GUCY2C-Targeted Chimeric Antigen Receptor (CAR)-Expressing T Cells Eliminate Colorectal Cancer Metastases. Cancer Immunol Res. 2018; 6(5): 509-516.
7. O'Rourke D M, Nasrallah M P, Desai A, et al. A single dose of peripherally infused EGFRvIII-directed CAR T cells mediates antigen loss and induces adaptive resistance in patients with recurrent glioblastoma. Sci Transl Med. 2017; 9(399).
8. Brown C E, Alizadeh D, Starr R, et al. Regression of Glioblastoma after Chimeric Antigen Receptor T-Cell Therapy. N Engl J Med. 2016; 375(26): 2561-9.
9. Ahmed N, Brawley V, Hegde M, et al. HER2-Specific Chimeric Antigen Receptor-Modified Virus-Specific T Cells for Progressive Glioblastoma: A Phase 1 Dose-Escalation Trial. JAMA Oncol. 2017; 3(8): 1094-1101.
10. Hambardzumyan D, Gutmann D H Kettenmann H. The role of microglia and macrophages in glioma maintenance and progression. Nat Neurosci. 2016; 19(1): 20-7.
11. Ravi V M, Neidert N, Will P, et al. T-cell dysfunction in the glioblastoma microenvironment is mediated by myeloid cells releasing interleukin-10. Nat Commun. 2022; 13(1): 925.
12. Lohr J, Ratliff T, Huppertz A, et al. Effector T-cell infiltration positively impacts survival of glioblastoma patients and is impaired by tumor-derived TGF-beta. Clin Cancer Res. 2011; 17(13): 4296-308.
13. Song E, Mao T, Dong H, et al. VEGF-C-driven lymphatic drainage enables immunosurveillance of brain tumours. Nature. 2020; 577(7792): 689-694.
14. Krenciute G, Krebs S, Torres D, et al. Characterization and Functional Analysis of scFv-based Chimeric Antigen Receptors to Redirect T Cells to IL13Ralpha2-positive Glioma. Mol Ther. 2016; 24(2): 354-363.
15. Krenciute G, Prinzing B L, Yi Z, et al. Transgenic Expression of IL15 Improves Antiglioma Activity of IL13Ralpha2-CAR T Cells but Results in Antigen Loss Variants. Cancer Immunol Res. 2017; 5(7): 571-581.
16. Brown C E, Aguilar B, Starr R, et al. Optimization of IL13Ralpha2-Targeted Chimeric Antigen Receptor T Cells for Improved Anti-tumor Efficacy against Glioblastoma. Mol Ther. 2018; 26(1): 31-44.
17. Pituch K C, Miska J, Krenciute G, et al. Adoptive Transfer of IL13Ralpha2-Specific Chimeric Antigen Receptor T Cells Creates a Pro-inflammatory Environment in Glioblastoma. Mol Ther. 2018; 26(4): 986-995.
18. Chmielewski M Abken H. CAR T Cells Releasing IL-18 Convert to T-Bet (high) FoxO1 (low) Effectors that Exhibit Augmented Activity against Advanced Solid Tumors. Cell Rep. 2017; 21(11): 3205-3219.
19. Chmielewski M, Kopecky C, Hombach A A Abken H. IL-12 release by engineered T cells expressing chimeric antigen receptors can effectively Muster an antigen-independent macrophage response on tumor cells that have shut down tumor antigen expression. Cancer Res. 2011; 71(17): 5697-706.
20. Shum T, Omer B, Tashiro H, et al. Constitutive Signaling from an Engineered IL7 Receptor Promotes Durable Tumor Elimination by Tumor-Redirected T Cells. Cancer Discov. 2017; 7(11): 1238-1247.
21. Zhao Z, Li Y, Liu W Li X. Engineered IL-7 Receptor Enhances the Therapeutic Effect of AXL-CAR-T Cells on Triple-Negative Breast Cancer. Biomed Res Int. 2020; 2020: 4795171.
22. Kagoya Y, Tanaka S, Guo T, et al. A novel chimeric antigen receptor containing a JAK-STAT signaling domain mediates superior antitumor effects. Nat Med. 2018; 24(3): 352-359.
23. Lanitis E, Rota G, Kosti P, et al. Optimized gene engineering of murine CAR-T cells reveals the beneficial effects of IL-15 coexpression. J Exp Med. 2021; 218(2).
24. Hurton L V, Singh H, Najjar A M, et al. Tethered IL-15 augments antitumor activity and promotes a stem-cell memory subset in tumor-specific T cells. Proc Natl Acad Sci USA. 2016; 113(48): E7788-E7797.
25. Ye X Z, Xu S L, Xin Y H, et al. Tumor-associated microglia/macrophages enhance the invasion of glioma stem-like cells via TGF-beta1 signaling pathway. J Immunol. 2012; 189(1): 444-53.
26. Pinton L, Masetto E, Vettore M, et al. The immune suppressive microenvironment of human gliomas depends on the accumulation of bone marrow-derived macrophages in the center of the lesion. J Immunother Cancer. 2019; 7(1): 58.
27. Balyasnikova I V, Wainwright D A, Solomaha E, et al. Characterization and immunotherapeutic implications for a novel antibody targeting interleukin (IL)-13 receptor alpha2. J Biol Chem. 2012; 287(36): 30215-27.
28. Kim J W, Young J S, Solomaha E, et al. A novel single-chain antibody redirects adenovirus to IL13Ralpha2-expressing brain tumors. Sci Rep. 2015; 5: 18133.
29. Jumper J, Evans R, Pritzel A, et al. Highly accurate protein structure prediction with AlphaFold. Nature. 2021; 596(7873): 583-589.
30. Chirifu M, Hayashi C, Nakamura T, et al. Crystal structure of the IL-15-IL-15Ralpha complex, a cytokine-receptor unit presented in trans. Nat Immunol. 2007; 8(9): 1001-7.
31. Dubois S, Mariner J, Waldmann T A Tagaya Y. IL-15Ralpha recycles and presents IL-15 In trans to neighboring cells. Immunity. 2002; 17(5): 537-47.
32. Lisi L, Ciotti G M, Braun D, et al. Expression of iNOS, CD163 and ARG-1 taken as M1 and M2 markers of microglial polarization in human glioblastoma and the surrounding normal parenchyma. Neurosci Lett. 2017; 645: 106-112.
33. Miska J, Rashidi A, Lee-Chang C, et al. Polyamines drive myeloid cell survival by buffering intracellular pH to promote immunosuppression in glioblastoma. Sci Adv. 2021; 7(8).
34. Bergamaschi C, Bear J, Rosati M, et al. Circulating IL-15 exists as heterodimeric complex with soluble IL-15Ralpha in human and mouse serum. Blood. 2012; 120(1): e1-8.
35. Chertova E, Bergamaschi C, Chertov O, et al. Characterization and favorable in vivo properties of heterodimeric soluble IL-15.IL-15Ralpha cytokine compared to IL-15 monomer. J Biol Chem. 2013; 288(25): 18093-103.
36. Batra S A, Rathi P, Guo L, et al. Glypican-3-Specific CAR T Cells Coexpressing IL15 and IL21 Have Superior Expansion and Antitumor Activity against Hepatocellular Carcinoma. Cancer Immunol Res. 2020; 8(3): 309-320.
37. De Leo A, Ugolini A Veglia F. Myeloid Cells in Glioblastoma Microenvironment. Cells. 2020; 10(1).
38. Sauer T, Parikh K, Sharma S, et al. CD70-specific CAR T cells have potent activity against acute myeloid leukemia without HSC toxicity. Blood. 2021; 138(4): 318-330.
39. Lee-Chang C, Rashidi A, Miska J, et al. Myeloid-Derived Suppressive Cells Promote B cell-Mediated Immunosuppression via Transfer of PD-L1 in Glioblastoma. Cancer Immunol Res. 2019; 7(12): 1928-1943.
40. Edin S, Kaprio T, Hagstrom J, et al. The Prognostic Importance of CD20 (+) B lymphocytes in Colorectal Cancer and the Relation to Other Immune Cell subsets. Sci Rep. 2019; 9(1): 19997.
41. Zhang Y, Yin X, Wang Q, et al. A novel gene expression signature-based on B-cell proportion to predict prognosis of patients with lung adenocarcinoma. BMC Cancer. 2021; 21(1): 1098.
42. Nielsen J S, Sahota R A, Milne K, et al. CD20+ tumor-infiltrating lymphocytes have an atypical CD27-memory phenotype and together with CD8⁺ T cells promote favorable prognosis in ovarian cancer. Clin Cancer Res. 2012; 18(12): 3281-92.
43. Ren F, Zhao Q, Huang L, et al. The R132H mutation in IDH1 promotes the recruitment of NK cells through CX3CL1/CX3CR1 chemotaxis and is correlated with a better prognosis in gliomas. Immunol Cell Biol. 2019; 97(5): 457-469.
44. Vauleon E, Tony A, Hamlat A, et al. Immune genes are associated with human glioblastoma pathology and patient survival. BMC Med Genomics. 2012; 5: 41.
45. Rodriguez-Garcia A, Lynn R C, Poussin M, et al. CAR-T cell-mediated depletion of immunosuppressive tumor-associated macrophages promotes endogenous antitumor immunity and augments adoptive immunotherapy. Nat Commun. 2021; 12(1): 877.
46. Ruella M, Klichinsky M, Kenderian S S, et al. Overcoming the Immunosuppressive Tumor Microenvironment of Hodgkin Lymphoma Using Chimeric Antigen Receptor T Cells. Cancer Discov. 2017; 7(10): 1154-1167.
47. Brown C E, Badie B, Barish M E, et al. Bioactivity and Safety of IL13Ralpha2-Redirected Chimeric Antigen Receptor CD8+ T Cells in Patients with Recurrent Glioblastoma. Clin Cancer Res. 2015; 21(18): 4062-72.
48. Vitanza N A, Johnson A J, Wilson A L, et al. Locoregional infusion of HER2-specific CAR T cells in children and young adults with recurrent or refractory CNS tumors: an interim analysis. Nat Med. 2021; 27(9): 1544-1552.
49. Hegde M, Mukherjee M, Grada Z, et al. Tandem CAR T cells targeting HER2 and IL13Ralpha2 mitigate tumor antigen escape. J Clin Invest. 2016; 126(8): 3036-52.
50. Bielamowicz K, Fousek K, Byrd T T, et al. Trivalent CAR T cells overcome interpatient antigenic variability in glioblastoma. Neuro Oncol. 2018; 20(4): 506-518.
51. Choi B D, Yu X, Castano A P, et al. CAR-T cells secreting BiTEs circumvent antigen escape without detectable toxicity. Nat Biotechnol. 2019; 37(9): 1049-1058.
52. Yin Y, Rodriguez J L, Li N, et al. Locally secreted BiTEs complement CAR T cells by enhancing killing of antigen heterogeneous solid tumors. Mol Ther. 2022; 30(7): 2537-2553.
53. Hu B, Zou Y, Zhang L, et al. Nucleofection with Plasmid DNA for CRISPR/Cas9-Mediated Inactivation of Programmed Cell Death Protein 1 in CD133-Specific CAR T Cells. Hum Gene Ther. 2019; 30(4): 446-458.
54. Prinzing B, Zebley C C, Petersen C T, et al. Deleting DNMT3A in CAR T cells prevents exhaustion and enhances antitumor activity. Sci Transl Med. 2021; 13(620): eabh0272.
55. Zhang Y, Zhang X, Cheng C, et al. CRISPR-Cas9 mediated LAG-3 disruption in CAR-T cells. Front Med. 2017; 11(4): 554-562.
56. Zhang W, Shi L, Zhao Z, et al. Disruption of CTLA-4 expression on peripheral blood CD8+ T cell enhances anti-tumor efficacy in bladder cancer. Cancer Chemother Pharmacol. 2019; 83(5): 911-920.

SEQUENCES
Mouse CAR.IL15f (mIL15 with (Gly4S)3 linker_pRV2011(M).
m47.CD28.CD3zeta)
SEQ ID NO: 1
ATG AAA ATT TTG AAA CCA TAT ATG AGG AAT ACA TCC ATC TCG TGC TAC TTG
TGT TTC CTT CTA AAC AGT CAC TTT TTA ACT GAG GCT GGC ATT CAT GTC TTC
ATT TTG GGC TGT GTC AGT GTA GGT CTC CCT AAA ACA GAG GCC AAC TGG ATA
GAT GTA AGA TAT GAC CTG GAG AAA ATT GAA AGC CTT ATT CAA TCT ATT CAT
ATT GAC ACC ACT TTA TAC ACT GAC AGT GAC TTT CAT CCC AGT TGC AAA GTT
ACT GCA ATG AAC TGC TTT CTC CTG GAA TTG CAG GTT ATT TTA CAT GAG TAC
AGT AAC ATG ACT CTT AAT GAA ACA GTA AGA AAC GTG CTC TAC CTT GCA AAC
AGC ACT CTG TCT TCT AAC AAG AAT GTA GCA GAA TCT GGC TGC AAG GAA TGT
GAG GAG CTG GAG GAG AAA ACC TTC ACA GAG TTT TTG CAA AGC TTT ATA CGC
ATT GTC CAA ATG TTC ATC AAC ACG TCC GGT GGC GGT GGC TCC GGC GGT GGT
GGG TCG GGT GGC GGC GGA TCT CAG GTG CAG CTG CAG CAG CCT GGC GCT GAA
CTC GTG CGG CCA GGC GCT TCT GTG AAG CTG AGC TGT AAA GCC AGC GGC TAC
ACC TTC AGC AAC TAC CTG ATG AAC TGG GTC AAG CAG CGG CCC GAG CAG GAC
CTG GAT TGG ATC GGC AGA ATC GAC CCC TAC GAC GGC GAC ATC GAC TAC AAC
CAG AAC TTC AAG GAC AAG GCC ATC CTG ACC GTG GAC AAG AGC AGC AGC ACC
GCC TAC ATG CAG CTG TCC AGC CTG ACC AGC GAG GAC AGC GCC GTG TAC TAC
TGC GCC AGA GGC TAC GGC ACA GCC TAC GGC GTG GAC TAT TGG GGC CAG GGC
ACA AGC GTG ACC GTG TCC AGC GCC AAG ACC ACC CCC CCT AAG CTG GAA GAG
GGC GAG TTC TCC GAG GCC CGG GTG GAC ATT GTG CTG ACA CAG TCT CCA GCC
AGC CTG GCC GTG TCC CTG GGA CAG AGA GCC ACC ATC AGC TGT AGG GCC AGC
GAG AGC GTG GAC AAC TAC GGC ATC AGC TTC ATG AAT TGG TTC CAG CAG AAG
CCC GGC CAG CCC CCC AAG CTG CTG ATC TAT GCC GCC AGC AGA CAG GGC AGC
GGA GTG CCT GCC AGA TTT TCT GGC AGC GGC TCC GGC ACC GAC TTC AGC CTG
AAC ATC CAC CCT ATG GAA GAG GAC GAC ACC GCC ATG TAC TTT TGC CAG CAG
AGC AAA GAG GTG CCC TGG ACC TTT GGC GGA GGC ACC AAG CTG GAA ATC AAG
CTG GAG GAT CCC AAA GTG CCC AGG GAT TGT GGT TGT AAG CCT TGC ATA TGT
ACA GTC CCA GAA GTA TTC TGG GCT CTG GTG GTG GTG GCC GGC GTG CTG TTT
TGT TAC GGC CTG CTC GTG ACA GTG GCC CTG TGC GTG ATC TGG ACC AAC AGC
AGA AGA AAC AGA GGC GGC CAG TCC GAC TAC ATG AAC ATG ACC CCC CGC AGA
CCC GGC CTG ACC AGA AAG CCT TAC CAG CCT TAC GCC CCT GCC AGA GAT TTC
GCC GCC TAC AGA CCC AGA GCC AAG TTC AGC AGA AGC GCC GAG ACA GCC GCC
AAC CTG CAG GAC CCT AAC CAG CTG TTC AAC GAG CTG AAC CTG GGC AGA CGG
GAA GAG TTC GAC GTG CTG GAA AAG AAG AGA GCC AGG GAC CCC GAG ATG GGC
GGC AAG CAG CAG AGA AGA AGA AAC CCC CAG GAA GGC GTG TAC AAC GCC CTG
CAG AAA GAC AAG ATG GCC GAG GCC TAC AGC GAG ATC GGC ACA AAG GGC GAG
AGA AGG CGG GGC AAG GGA CAC GAT GGC CTG TTC CAG GGC CTG AGC ACC GCC
ACC AAG GAC ACA TTC GAT GCC CTG CAC ATG CAG ACA CTG GCC CCC AGA TGA
Mouse CAR.IL15f (mIL15 with (Gly4S)3 linker_pRV2011(M).
m47.CD28.CD3zeta)
SEQ ID NO: 2
MKILKPYMRNTSISCYLCFLLNSHFLTEAGIHVFILGCVSVGLPKTEANWIDVRYDLEKIESLIQSI
HIDTTLYTDSDFHPSCKVTAMNCFLLELQVILHEYSNMTLNETVRNVLYLANSTLSSNKNVAESGCK
ECEELEEKTFTEFLQSFIRIVQMFINTSGGGGSGGGGSGGGGSQVQLQQPGAELVRPGASVKLSCKA
SGYTFSNYLMNWVKQRPEQDLDWIGRIDPYDGDIDYNQNFKDKAILTVDKSSSTAYMQLSSLTSEDS
AVYYCARGYGTAYGVDYWGQGTSVTVSSAKTTPPKLEEGEFSEARVDIVLTQSPASLAVSLGQRATI
SCRASESVDNYGISFMNWFQQKPGQPPKLLIYAASRQGSGVPARFSGSGSGTDFSLNIHPMEEDDTA
MYFCQQSKEVPWTFGGGTKLEIKLEDPKVPRDCGCKPCICTVPEVFWALVVVAGVLFCYGLLVTVAL
CVIWINSRRNRGGQSDYMNMTPRRPGLTRKPYQPYAPARDFAAYRPRAKFSRSAETAANLQDPNQLF
NELNLGRREEFDVLEKKRARDPEMGGKQQRRRNPQEGVYNALQKDKMAEAYSEIGTKGERRRGKGHD
GLFQGLSTATKDTFDALHMQTLAPR*
(*stop codon)
mIL15 with signal peptide sequence
SEQ ID NO: 3
ATG AAA ATT TTG AAA CCA TAT ATG AGG AAT ACA TCC ATC TCG TGC TAC TTG
TGT TTC CTT CTA AAC AGT CAC TTT TTA ACT GAG GCT GGC ATT CAT GTC TTC
ATT TTG GGC TGT GTC AGT GTA GGT CTC CCT AAA ACA GAG GCC AAC TGG ATA
GAT GTA AGA TAT GAC CTG GAG AAA ATT GAA AGC CTT ATT CAA TCT ATT CAT
ATT GAC ACC ACT TTA TAC ACT GAC AGT GAC TTT CAT CCC AGT TGC AAA GTT
ACT GCA ATG AAC TGC TTT CTC CTG GAA TTG CAG GTT ATT TTA CAT GAG TAC
AGT AAC ATG ACT CTT AAT GAA ACA GTA AGA AAC GTG CTC TAC CTT GCA AAC
AGC ACT CTG TCT TCT AAC AAG AAT GTA GCA GAA TCT GGC TGC AAG GAA TGT
GAG GAG CTG GAG GAG AAA ACC TTC ACA GAG TTT TTG CAA AGC TTT ATA CGC
ATT GTC CAA ATG TTC ATC AAC ACG TCC
mIL15 with signal peptide
SEQ ID NO: 4
MKILKPYMRNTSISCYLCFLLNSHFLTEAGIHVFILGCVSVGLPKTEANWIDVRYDLEKIESLIQSI
HIDTTLYTDSDFHPSCKVTAMNCFLLELQVILHEYSNMTLNETVRNVLYLANSTLSSNKNVAESGCK
ECEELEEKTFTEFLQSFIRIVQMFINTS
(Gly4S)₃ linker
SEQ ID NO: 5
GGTGGCGGTGGCTCCGGCGGTGGTGGGTCGGGTGGCGGCGGATCT
Mouse scFv47 (IL13Rα2 scFv47)
SEQ ID NO: 6
CAG GTG CAG CTG CAG CAG CCT GGC GCT GAA CTC GTG CGG CCA GGC GCT TCT
GTG AAG CTG AGC TGT AAA GCC AGC GGC TAC ACC TTC AGC AAC TAC CTG ATG
AAC TGG GTC AAG CAG CGG CCC GAG CAG GAC CTG GAT TGG ATC GGC AGA ATC
GAC CCC TAC GAC GGC GAC ATC GAC TAC AAC CAG AAC TTC AAG GAC AAG GCC
ATC CTG ACC GTG GAC AAG AGC AGC AGC ACC GCC TAC ATG CAG CTG TCC AGC
CTG ACC AGC GAG GAC AGC GCC GTG TAC TAC TGC GCC AGA GGC TAC GGC ACA
GCC TAC GGC GTG GAC TAT TGG GGC CAG GGC ACA AGC GTG ACC GTG TCC AGC
GCC AAG ACC ACC CCC CCT AAG CTG GAA GAG GGC GAG TTC TCC GAG GCC CGG
GTG GAC ATT GTG CTG ACA CAG TCT CCA GCC AGC CTG GCC GTG TCC CTG GGA
CAG AGA GCC ACC ATC AGC TGT AGG GCC AGC GAG AGC GTG GAC AAC TAC GGC
ATC AGC TTC ATG AAT TGG TTC CAG CAG AAG CCC GGC CAG CCC CCC AAG CTG
CTG ATC TAT GCC GCC AGC AGA CAG GGC AGC GGA GTG CCT GCC AGA TTT TCT
GGC AGC GGC TCC GGC ACC GAC TTC AGC CTG AAC ATC CAC CCT ATG GAA GAG
GAC GAC ACC GCC ATG TAC TTT TGC CAG CAG AGC AAA GAG GTG CCC TGG ACC
TTT GGC GGA GGC ACC AAG CTG GAA ATC AAG CTG GAG
Mouse scFv47 (IL13Rα2 scFv47)
SEQ ID NO: 7
QVQLQQPGAELVRPGASVKLSCKASGYTFSNYLMNWVKQRPEQDLDWIGRIDPYDGDIDYNQNFKDK
AILTVDKSSSTAYMQLSSLTSEDSAVYYCARGYGTAYGVDYWGQGTSVTVSSAKTTPPKLEEGEFSE
ARVDIVLTQSPASLAVSLGQRATISCRASESVDNYGISFMNWFQQKPGQPPKLLIYAASRQGSGVPA
RFSGSGSGTDFSLNIHPMEEDDTAMYFCQQSKEVPWTFGGGTKLEIKLE
Mouse CAR.IL15s (m47CD28CAR T2A mIL15)
SEQ ID NO: 8
ATG GGC GTG CCC ACC CAG CTG CTG GGA CTG CTG CTG CTG TGG ATC ACC GAC
GCC ATC TGC CAG GTG CAG CTG CAG CAG CCT GGC GCT GAA CTC GTG CGG CCA
GGC GCT TCT GTG AAG CTG AGC TGT AAA GCC AGC GGC TAC ACC TTC AGC AAC
TAC CTG ATG AAC TGG GTC AAG CAG CGG CCC GAG CAG GAC CTG GAT TGG ATC
GGC AGA ATC GAC CCC TAC GAC GGC GAC ATC GAC TAC AAC CAG AAC TTC AAG
GAC AAG GCC ATC CTG ACC GTG GAC AAG AGC AGC AGC ACC GCC TAC ATG CAG
CTG TCC AGC CTG ACC AGC GAG GAC AGC GCC GTG TAC TAC TGC GCC AGA GGC
TAC GGC ACA GCC TAC GGC GTG GAC TAT TGG GGC CAG GGC ACA AGC GTG ACC
GTG TCC AGC GCC AAG ACC ACC CCC CCT AAG CTG GAA GAG GGC GAG TTC TCC
GAG GCC CGG GTG GAC ATT GTG CTG ACA CAG TCT CCA GCC AGC CTG GCC GTG
TCC CTG GGA CAG AGA GCC ACC ATC AGC TGT AGG GCC AGC GAG AGC GTG GAC
AAC TAC GGC ATC AGC TTC ATG AAT TGG TTC CAG CAG AAG CCC GGC CAG CCC
CCC AAG CTG CTG ATC TAT GCC GCC AGC AGA CAG GGC AGC GGA GTG CCT GCC
AGA TTT TCT GGC AGC GGC TCC GGC ACC GAC TTC AGC CTG AAC ATC CAC CCT
ATG GAA GAG GAC GAC ACC GCC ATG TAC TTT TGC CAG CAG AGC AAA GAG GTG
CCC TGG ACC TTT GGC GGA GGC ACC AAG CTG GAA ATC AAG CTG GAG GAT CCC
AAA GTG CCC AGG GAT TGT GGT TGT AAG CCT TGC ATA TGT ACA GTC CCA GAA
GTA TTC TGG GCT CTG GTG GTG GTG GCC GGC GTG CTG TTT TGT TAC GGC CTG
CTC GTG ACA GTG GCC CTG TGC GTG ATC TGG ACC AAC AGC AGA AGA AAC AGA
GGC GGC CAG TCC GAC TAC ATG AAC ATG ACC CCC CGC AGA CCC GGC CTG ACC
AGA AAG CCT TAC CAG CCT TAC GCC CCT GCC AGA GAT TTC GCC GCC TAC AGA
CCC AGA GCC AAG TTC AGC AGA AGC GCC GAG ACA GCC GCC AAC CTG CAG GAC
CCT AAC CAG CTG TTC AAC GAG CTG AAC CTG GGC AGA CGG GAA GAG TTC GAC
GTG CTG GAA AAG AAG AGA GCC AGG GAC CCC GAG ATG GGC GGC AAG CAG CAG
AGA AGA AGA AAC CCC CAG GAA GGC GTG TAC AAC GCC CTG CAG AAA GAC AAG
ATG GCC GAG GCC TAC AGC GAG ATC GGC ACA AAG GGC GAG AGA AGG CGG GGC
AAG GGA CAC GAT GGC CTG TTC CAG GGC CTG AGC ACC GCC ACC AAG GAC ACA
TTC GAT GCC CTG CAC ATG CAG ACA CTG GCC CCC AGA TGC GGC CGC GAG GGC
AGA GGA AGT CTT CTA ACA TGC GGT GAC GTG GAG GAG AAT CCC GGC CCT AGG
ATG AAA ATT TTG AAA CCA TAT ATG AGG AAT ACA TCC ATC TCG TGC TAC TTG
TGT TTC CTT CTA AAC AGT CAC TTT TTA ACT GAG GCT GGC ATT CAT GTC TTC
ATT TTG GGC TGT GTC AGT GTA GGT CTC CCT AAA ACA GAG GCC AAC TGG ATA
GAT GTA AGA TAT GAC CTG GAG AAA ATT GAA AGC CTT ATT CAA TCT ATT CAT
ATT GAC ACC ACT TTA TAC ACT GAC AGT GAC TTT CAT CCC AGT TGC AAA GTT
ACT GCA ATG AAC TGC TTT CTC CTG GAA TTG CAG GTT ATT TTA CAT GAG TAC
AGT AAC ATG ACT CTT AAT GAA ACA GTA AGA AAC GTG CTC TAC CTT GCA AAC
AGC ACT CTG TCT TCT AAC AAG AAT GTA GCA GAA TCT GGC TGC AAG GAA TGT
GAG GAG CTG GAG GAG AAA ACC TTC ACA GAG TTT TTG CAA AGC TTT ATA CGC
ATT GTC CAA ATG TTC ATC AAC ACG TCC TGA
Mouse CAR.IL15s (m47CD28.CD3zeta CAR T2A mIL15)
SEQ ID NO: 9
MGVPTQLLGLLLLWITDAICQVQLQQPGAELVRPGASVKLSCKASGYTFSNYLMNWVKQRPEQDLDW
IGRIDPYDGDIDYNQNFKDKAILTVDKSSSTAYMQLSSLTSEDSAVYYCARGYGTAYGVDYWGQGTS
VTVSSAKTTPPKLEEGEFSEARVDIVLTQSPASLAVSLGQRATISCRASESVDNYGISFMNWFQQKP
GQPPKLLIYAASRQGSGVPARFSGSGSGTDFSLNIHPMEEDDTAMYFCQQSKEVPWTFGGGTKLEIK
LEDPKVPRDCGCKPCICTVPEVFWALVVVAGVLFCYGLLVTVALCVIWTNSRRNRGGQSDYMNMTPR
RPGLTRKPYQPYAPARDFAAYRPRAKFSRSAETAANLQDPNQLFNELNLGRREEFDVLEKKRARDPE
MGGKQQRRRNPQEGVYNALQKDKMAEAYSEIGTKGERRRGKGHDGLFQGLSTATKDTFDALHMQTLA
PRCGREGRGSLLTCGDVEENPGPRMKILKPYMRNTSISCYLCFLLNSHFLTEAGIHVFILGCVSVGL
PKTEANWIDVRYDLEKIESLIQSIHIDTTLYTDSDFHPSCKVTAMNCFLLELQVILHEYSNMTLNET
VRNVLYLANSTLSSNKNVAESGCKECEELEEKTFTEFLQSFIRIVQMFINTS*
(*stop codon)
Human IL15Rα Forward Primer
SEQ ID NO: 10
GTGGCCCTGTGGATACACAC
Human IL15Rα Reverse Primer
SEQ ID NO: 11
ACAACAGCAGCTATTGTCCCG
Mouse IL15Rα Forward Primer
SEQ ID NO: 12
AATCAGATACCGCAATGACCAC
Mouse IL15Rα Reverse Primer
SEQ ID NO: 13
CAGAAGTTGTTTGGGATGGTGT
β-Actin Forward Primer
SEQ ID NO: 14
TTGCTGACAGGATGCAGAAG
β-Actin Reverse Primer
SEQ ID NO: 15
TTGCTGACAGGATGCAGAAG
Signal peptide
SEQ ID NO: 16
MKILKPYMRNTSISCYLCFLLNSHFLTEA
CD28.CD23 zeta (BamHl restriction site, mouse IgG
hinge, CD28.CD3zeta)
SEQ ID NO: 17
DPKVPRDCGCKPCICTVPEVFWALVVVAGVLFCYGLLVTVALCVIWTNSRRNRGGQSDYMNMTPRRP
GLTRKPYQPYAPARDFAAYRPRAKFSRSAETAANLQDPNQLFNELNLGRREEFDVLEKKRARDPEMG
GKQQRRRNPQEGVYNALQKDKMAEAYSEIGTKGERRRGKGHDGLFQGLSTATKDTFDALHMQTLAPR
T2A
SEQ ID NO: 18
EGRGSLLTCGDVEENPGP
Human IL15 (NCBI Ref Seq NM_000585.5) with signal
peptide sequence
SEQ ID NO: 19
ATGAGAATTTCGAAACCACATTTGAGAAGTATTTCCATCCAGTGCTACTTGTGTTTACTTCTAAACA
GTCATTTTCTAACTGAAGCTGGCATTCATGTCTTCATTTTGGGCTGTTTCAGTGCAGGGCTTCCTAA
AACAGAAGCCAACTGGGTGAATGTAATAAGTGATTTGAAAAAAATTGAAGATCTTATTCAATCTATG
CATATTGATGCTACTTTATATACGGAAAGTGATGTTCACCCCAGTTGCAAAGTAACAGCAATGAAGT
GCTTTCTCTTGGAGTTACAAGTTATTTCACTTGAGTCCGGAGATGCAAGTATTCATGATACAGTAGA
AAATCTGATCATCCTAGCAAACAACAGTTTGTCTTCTAATGGGAATGTAACAGAATCTGGATGCAAA
GAATGTGAGGAACTGGAGGAAAAAAATATTAAAGAATTTTTGCAGAGTTTTGTACATATTGTCCAAA
TGTTCATCAACACTTCTTGA
Human IL15 with signal peptide
SEQ ID NO: 20
MRISKPHLRSISIQCYLCLLLNSHFLTEAGIHVFILGCFSAGLPKTEANWVNVISDLKKIEDLIQSM
HIDATLYTESDVHPSCKVTAMKCFLLELQVISLESGDASIHDTVENLIILANNSLSSNGNVTESGCK
ECEELEEKNIKEFLQSFVHIVQMFINTS
(Gly4S)₃ linker
SEQ ID NO: 21
GGGGS GGGGS GGGGS
Humanized scFv47 IL13Rα2 (clone 47)
SEQ ID NO: 22
CAGGTGCAGCTGGTGCAGTCCGGAGCAGAGGTGAAGAAGCCAGGAAGCTCCGTGAAGGTGTCTTGCA
AGGCCAGCGGCTATACCTTCTCTAACTACCTGATGAATTGGGTGAGGCAGGCACCAGGACAGGGACT
GGAGTGGATGGGCCGCATCGATCCTTATGACGGCGATATCGACTACAACCAGAACTTCAAGGATCGG
GTGACCATCACAGCCGACAAGAGCACCTCCACAGCCTATATGGAGCTGTCTAGCCTGCGGAGCGAGG
ATACCGCCGTGTACTATTGTGCCAGAGGCTACGGCACAGCCTATGGAGTGGACTACTGGGGACAGGG
CACCCTGGTGACAGTGTCCTCTGGTGGCGGTGGCTCCGGCGGTGGTGGGTCGGGTGGCGGCGGATCT
GACATCGTGATGACCCAGTCCCCTGATTCTCTGGCCGTGTCTCTGGGAGAGAGGGCAACAATCAACT
GCAGAGCCTCTGAGAGCGTGGACAACTACGGCATCAGCTTCATGAATTGGTATCAGCAGAAGCCAGG
CCAGCCCCCTAAGCTGCTGATCTACGCAGCAAGCAGGCAGGGATCCGGAGTGCCCGACCGGTTCTCC
GGATCTGGAAGCGGAACCGACTTCACCCTGACAATCAGCTCCCTGCAGGCCGAGGATGTGGCCGTGT
ACTATTGTCAGCAGTCCAAGGAGGTGCCATGGACCTTTGGCGGAGGAACAAAGCTGGAGATCAAG
Humanized scFv IL13Rα2 (clone 47)
SEQ ID NO: 23
QVQLVQSGAEVKKPGSSVKVSCKASGYTFSNYLMNWVRQAPGQGLEWMGRIDPYDGDIDYNQNFKDR
VTITADKSTSTAYMELSSLRSEDTAVYYCARGYGTAYGVDYWGQGTLVTVSSGGGGSGGGGSGGGGS
DIVMTQSPDSLAVSLGERATINCRASESVDNYGISFMNWYQQKPGQPPKLLIYAASRQGSGVPDRFS
GSGSGTDFTLTISSLQAEDVAVYYCQQSKEVPWTFGGGTKLEIK
Part of CAR sequence downstream of scFv IL13Ra2
(clone 47) Human IgG1 hinge. CD28, CD3zeta
SEQ ID NO: 24
DLEPKSCDKTHTCPPCPDPKFWVLVVVGGVLACYSLLVTVAFIIFWVRSKRSRLLHSDYMNMTPRRP
GPTRKHYQPYAPPRDFAAYRSRVKFSRSADAPAYQQGONQLYNELNLGRREEYDVLDKRRGRDPEMG
GKPRRKNPQEGLYNELQKDKMAEAYSEIGMKGERRRGKGHDGLYQGLSTATKDTYDALHMQALPPR
Part of CAR sequence downstream of scFv IL13Ra2
(clone 47) Human IgG1 hinge, CD28, CD3zeta
SEQ ID NO: 25
GATCTCGAGCCCAAATCTTGTGACAAAACTCACACATGCCCACCGTGCCCGGATCCCAAATTTTGGG
TGCTGGTGGTGGTTGGTGGAGTCCTGGCTTGCTATAGCTTGCTAGTAACAGTGGCCTTTATTATTTT
CTGGGTGAGGAGTAAGAGGAGCAGGCTCCTGCACAGTGACTACATGAACATGACTCCCCGCCGCCCC
GGGCCCACCCGCAAGCATTACCAGCCCTATGCCCCACCACGCGACTTCGCAGCCTATCGCTCCAGAG
TGAAGTTCAGCAGGAGCGCAGACGCCCCCGCGTACCAGCAGGGCCAGAACCAGCTCTATAACGAGCT
CAATCTAGGACGAAGAGAGGAGTACGATGTTTTGGACAAGAGACGTGGCCGGGACCCTGAGATGGGG
GGAAAGCCGAGAAGGAAGAACCCTCAGGAAGGCCTGTACAATGAACTGCAGAAAGATAAGATGGCGG
AGGCCTACAGTGAGATTGGGATGAAAGGCGAGCGCCGGAGGGGCAAGGGGCACGATGGCCTTTACCA
GGGTCTCAGTACAGCCACCAAGGACACCTACGACGCCCTTCACATGCAGGCCCTGCCCCCTCGC
Humanized CAR.IL15f with signal peptide
SEQ ID NO: 26
MRISKPHLRSISIQCYLCLLLNSHFLTEAGIHVFILGCFSAGLPKTEANWVNVISDLKKIEDLIQSM
HIDATLYTESDVHPSCKVTAMKCFLLELQVISLESGDASIHDTVENLIILANNSLSSNGNVTESGCK
ECEELEEKNIKEFLQSFVHIVQMFINTSGGGGSGGGGSGGGGSQVQLVQSGAEVKKPGSSVKVSCKA
SGYTFSNYLMNWVRQAPGQGLEWMGRIDPYDGDIDYNQNFKDRVTITADKSTSTAYMELSSLRSEDT
AVYYCARGYGTAYGVDYWGQGTLVTVSSGGGGSGGGGSGGGGSDIVMTQSPDSLAVSLGERATINCR
ASESVDNYGISFMNWYQQKPGQPPKLLIYAASRQGSGVPDRFSGSGSGTDFTLTISSLQAEDVAVYY
CQQSKEVPWTFGGGTKLEIKDLEPKSCDKTHTCPPCPDPKFWVLVVVGGVLACYSLLVTVAFIIFWV
RSKRSRLLHSDYMNMTPRRPGPTRKHYQPYAPPRDFAAYRSRVKFSRSADAPAYQQGQNQLYNELNL
GRREEYDVLDKRRGRDPEMGGKPRRKNPQEGLYNELQKDKMAEAYSEIGMKGERRRGKGHDGLYQGL
STATKDTYDALHMQALPPR
Humanized CAR.IL15f with signal peptide sequence
SEQ ID NO: 27
ATGAGAATTTCGAAACCACATTTGAGAAGTATTTCCATCCAGTGCTACTTGTGTTTACTTCTAAACA
GTCATTTTCTAACTGAAGCTGGCATTCATGTCTTCATTTTGGGCTGTTTCAGTGCAGGGCTTCCTAA
AACAGAAGCCAACTGGGTGAATGTAATAAGTGATTTGAAAAAAATTGAAGATCTTATTCAATCTATG
CATATTGATGCTACTTTATATACGGAAAGTGATGTTCACCCCAGTTGCAAAGTAACAGCAATGAAGT
GCTTTCTCTTGGAGTTACAAGTTATTTCACTTGAGTCCGGAGATGCAAGTATTCATGATACAGTAGA
AAATCTGATCATCCTAGCAAACAACAGTTTGTCTTCTAATGGGAATGTAACAGAATCTGGATGCAAA
GAATGTGAGGAACTGGAGGAAAAAAATATTAAAGAATTTTTGCAGAGTTTTGTACATATTGTCCAAA
TGTTCATCAACACTTCTGGTGGCGGTGGCTCCGGCGGTGGTGGGTCGGGTGGCGGCGGATCTCAGGT
GCAGCTGGTGCAGTCCGGAGCAGAGGTGAAGAAGCCAGGAAGCTCCGTGAAGGTGTCTTGCAAGGCC
AGCGGCTATACCTTCTCTAACTACCTGATGAATTGGGTGAGGCAGGCACCAGGACAGGGACTGGAGT
GGATGGGCCGCATCGATCCTTATGACGGCGATATCGACTACAACCAGAACTTCAAGGATCGGGTGAC
CATCACAGCCGACAAGAGCACCTCCACAGCCTATATGGAGCTGTCTAGCCTGCGGAGCGAGGATACC
GCCGTGTACTATTGTGCCAGAGGCTACGGCACAGCCTATGGAGTGGACTACTGGGGACAGGGCACCC
TGGTGACAGTGTCCTCTGGTGGCGGTGGCTCCGGCGGTGGTGGGTCGGGTGGCGGCGGATCTGACAT
CGTGATGACCCAGTCCCCTGATTCTCTGGCCGTGTCTCTGGGAGAGAGGGCAACAATCAACTGCAGA
GCCTCTGAGAGCGTGGACAACTACGGCATCAGCTTCATGAATTGGTATCAGCAGAAGCCAGGCCAGC
CCCCTAAGCTGCTGATCTACGCAGCAAGCAGGCAGGGATCCGGAGTGCCCGACCGGTTCTCCGGATC
TGGAAGCGGAACCGACTTCACCCTGACAATCAGCTCCCTGCAGGCCGAGGATGTGGCCGTGTACTAT
TGTCAGCAGTCCAAGGAGGTGCCATGGACCTTTGGCGGAGGAACAAAGCTGGAGATCAAGGATCTCG
AGCCCAAATCTTGTGACAAAACTCACACATGCCCACCGTGCCCGGATCCCAAATTTTGGGTGCTGGT
GGTGGTTGGTGGAGTCCTGGCTTGCTATAGCTTGCTAGTAACAGTGGCCTTTATTATTTTCTGGGTG
AGGAGTAAGAGGAGCAGGCTCCTGCACAGTGACTACATGAACATGACTCCCCGCCGCCCCGGGCCCA
CCCGCAAGCATTACCAGCCCTATGCCCCACCACGCGACTTCGCAGCCTATCGCTCCAGAGTGAAGTT
CAGCAGGAGCGCAGACGCCCCCGCGTACCAGCAGGGCCAGAACCAGCTCTATAACGAGCTCAATCTA
GGACGAAGAGAGGAGTACGATGTTTTGGACAAGAGACGTGGCCGGGACCCTGAGATGGGGGGAAAGC
CGAGAAGGAAGAACCCTCAGGAAGGCCTGTACAATGAACTGCAGAAAGATAAGATGGCGGAGGCCTA
CAGTGAGATTGGGATGAAAGGCGAGCGCCGGAGGGGCAAGGGGCACGATGGCCTTTACCAGGGTCTC
AGTACAGCCACCAAGGACACCTACGACGCCCTTCACATGCAGGCCCTGCCCCCTCGCTGA
Humanized CAR.IL15f without signal peptide
SEQ ID NO: 28
GIHVFILGCFSAGLPKTEANWVNVISDLKKIEDLIQSMHIDATLYTESDVHPSCKVTAMKCFLLELQ
VISLESGDASIHDTVENLIILANNSLSSNGNVTESGCKECEELEEKNIKEFLQSFVHIVQMFINTSG
GGGSGGGGSGGGGSQVQLVQSGAEVKKPGSSVKVSCKASGYTFSNYLMNWVRQAPGQGLEWMGRIDP
YDGDIDYNQNFKDRVTITADKSTSTAYMELSSLRSEDTAVYYCARGYGTAYGVDYWGQGTLVTVSSG
GGGSGGGGSGGGGSDIVMTQSPDSLAVSLGERATINCRASESVDNYGISFMNWYQQKPGQPPKLLIY
AASRQGSGVPDRFSGSGSGTDFTLTISSLQAEDVAVYYCQQSKEVPWTFGGGTKLEIKDLEPKSCDK
THTCPPCPDPKFWVLVVVGGVLACYSLLVTVAFIIFWVRSKRSRLLHSDYMNMTPRRPGPTRKHYQP
YAPPRDFAAYRSRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
Human IL15 (NCBI Ref Seq NM_000585.5)
without signal peptide sequence
SEQ ID NO: 29
GGCATTCATGTCTTCATTTTGGGCTGTTTCAGTGCAGGGCTTCCTAAAACAGAAGCCAACTGGGTGA
ATGTAATAAGTGATTTGAAAAAAATTGAAGATCTTATTCAATCTATGCATATTGATGCTACTTTATA
TACGGAAAGTGATGTTCACCCCAGTTGCAAAGTAACAGCAATGAAGTGCTTTCTCTTGGAGTTACAA
GTTATTTCACTTGAGTCCGGAGATGCAAGTATTCATGATACAGTAGAAAATCTGATCATCCTAGCAA
ACAACAGTTTGTCTTCTAATGGGAATGTAACAGAATCTGGATGCAAAGAATGTGAGGAACTGGAGGA
AAAAAATATTAAAGAATTTTTGCAGAGTTTTGTACATATTGTCCAAATGTTCATCAACACTTCTTGA
Human IL15 without signal peptide
SEQ ID NO: 30
GIHVFILGCFSAGLPKTEANWVNVISDLKKIEDLIQSMHIDATLYTESDVHPSCKVTAMKCFLLELQ
VISLESGDASIHDTVENLIILANNSLSSNGNVTESGCKECEELEEKNIKEFLQSFVHIVQMFINTS
Humanized scFv IL13Ra2 (clone 47) D55E mutant
SEQ ID NO: 31
QVQLVQSGAEVKKPGSSVKVSCKASGYTFSNYLMNWVRQAPGQGLEWMGRIDPYEGDIDYNQNFKDR
VTITADKSTSTAYMELSSLRSEDTAVYYCARGYGTAYGVDYWGQGTLVTVSSGGGGSGGGGSGGGGS
DIVMTQSPDSLAVSLGERATINCRASESVDNYGISFMNWYQQKPGQPPKLLIYAASRQGSGVPDRFS
GSGSGTDFTLTISSLQAEDVAVYYCQQSKEVPWTFGGGTKLEIK
Humanized scFv IL13Ra2 (clone 47) G56A mutant
SEQ ID NO: 32
QVQLVQSGAEVKKPGSSVKVSCKASGYTFSNYLMNWVRQAPGQGLEWMGRIDPYDADIDYNQNFKDR
VTITADKSTSTAYMELSSLRSEDTAVYYCARGYGTAYGVDYWGQGTLVTVSSGGGGSGGGGSGGGGS
DIVMTQSPDSLAVSLGERATINCRASESVDNYGISFMNWYQQKPGQPPKLLIYAASRQGSGVPDRFS
GSGSGTDFTLTISSLQAEDVAVYYCQQSKEVPWTFGGGTKLEIK
Humanized scFv IL13Ra2 (clone 47) D57E mutant
SEQ ID NO: 33
QVQLVQSGAEVKKPGSSVKVSCKASGYTFSNYLMNWVRQAPGQGLEWMGRIDPYDGEIDYNQNFKDR
VTITADKSTSTAYMELSSLRSEDTAVYYCARGYGTAYGVDYWGQGTLVTVSSGGGGSGGGGSGGGGS
DIVMTQSPDSLAVSLGERATINCRASESVDNYGISFMNWYQQKPGQPPKLLIYAASRQGSGVPDRFS
GSGSGTDFTLTISSLQAEDVAVYYCQQSKEVPWTFGGGTKLEIK
Humanized scFv IL13Ra2 (clone 47) D55E and
G56A mutant
SEQ ID NO: 34
QVQLVQSGAEVKKPGSSVKVSCKASGYTFSNYLMNWVRQAPGQGLEWMGRIDPYEADIDYNQNFKDR
VTITADKSTSTAYMELSSLRSEDTAVYYCARGYGTAYGVDYWGQGTLVTVSSGGGGSGGGGSGGGGS
DIVMTQSPDSLAVSLGERATINCRASESVDNYGISFMNWYQQKPGQPPKLLIYAASRQGSGVPDRFS
GSGSGTDFTLTISSLQAEDVAVYYCQQSKEVPWTFGGGTKLEIK
Humanized scFv IL13Ra2 (clone 47) D57E and
G56A mutant
SEQ ID NO: 35
QVQLVQSGAEVKKPGSSVKVSCKASGYTFSNYLMNWVRQAPGQGLEWMGRIDPYDAEIDYNQNFKDR
VTITADKSTSTAYMELSSLRSEDTAVYYCARGYGTAYGVDYWGQGTLVTVSSGGGGSGGGGSGGGGS
DIVMTQSPDSLAVSLGERATINCRASESVDNYGISFMNWYQQKPGQPPKLLIYAASRQGSGVPDRFS
GSGSGTDFTLTISSLQAEDVAVYYCQQSKEVPWTFGGGTKLEIK
Humanized IL15 scFv IL13Ra2 with signal
peptide
SEQ ID NO: 36
MRISKPHLRSISIQCYLCLLLNSHFLTEAGIHVFILGCFSAGLPKTEANWVNVISDLKKIEDLIQSM
HIDATLYTESDVHPSCKVTAMKCFLLELQVISLESGDASIHDTVENLIILANNSLSSNGNVTESGCK
ECEELEEKNIKEFLQSFVHIVQMFINTSGGGGSGGGGSGGGGSQVQLVQSGAEVKKPGSSVKVSCKA
SGYTFSNYLMNWVRQAPGQGLEWMGRIDPYDGDIDYNQNFKDRVTITADKSTSTAYMELSSLRSEDT
AVYYCARGYGTAYGVDYWGQGTLVTVSSGGGGSGGGGSGGGGSDIVMTQSPDSLAVSLGERATINCR
ASESVDNYGISFMNWYQQKPGQPPKLLIYAASRQGSGVPDRFSGSGSGTDFTLTISSLQAEDVAVYY
CQQSKEVPWTFGGGTKLEIK
Humanized IL15 scFv IL13Ra2 without signal
peptide
SEQ ID NO: 37
GIHVFILGCFSAGLPKTEANWVNVISDLKKIEDLIQSMHIDATLYTESDVHPSCKVTAMKCFLLELQ
VISLESGDASIHDTVENLIILANNSLSSNGNVTESGCKECEELEEKNIKEFLQSFVHIVQMFINTSG
GGGSGGGGSGGGGSQVQLVQSGAEVKKPGSSVKVSCKASGYTFSNYLMNWVRQAPGQGLEWMGRIDP
YDGDIDYNQNFKDRVTITADKSTSTAYMELSSLRSEDTAVYYCARGYGTAYGVDYWGQGTLVTVSSG
GGGSGGGGSGGGGSDIVMTQSPDSLAVSLGERATINCRASESVDNYGISFMNWYQQKPGQPPKLLIY
AASRQGSGVPDRFSGSGSGTDFTLTISSLQAEDVAVYYCQQSKEVPWTFGGGTKLEIK
Signal Peptide
SEQ ID NO: 38
MRISKPHLRSISIQCYLCLLLNSHFLTEA

What is claimed:

1. An engineered polynucleotide encoding a fusion protein, the fusion protein comprising:
(a) a chimeric antigen receptor comprising a single chain antibody to IL13Rα2 (scFv) and
(b) interleukin 15 (IL15).

2. The engineered polynucleotide of claim 1, wherein the scFv and the IL15 are separated by a linker.

3. The engineered polynucleotide of claim 2, wherein the linker is (Gly₄Ser)₃.

4. The engineered polynucleotide of any one of claims 1-3, wherein the chimeric antigen receptor further comprises a transmembrane domain, and wherein the scFv is between the IL15 and the transmembrane domain.

5. The engineered polynucleotide of claim 1, wherein the sequence encoding the scFv comprises a sequence having at least 95% identity to SEQ ID NO: 23.

6. The engineered polynucleotide of claim 1, wherein the sequence encoding the IL15 comprises a sequence having at least 95% identity to SEQ ID NO: 29.

7. The engineered polynucleotide of claim 1, wherein the fusion protein comprises a sequence having at least 95% sequence identity to SEQ ID NO: 37.

8. The engineered polynucleotide of any one of claims 1-7, wherein the polynucleotide is operably linked to an exogenous promoter sequence capable of expressing the polynucleotide in a host cell.

9. The engineered polynucleotide of any one of claims 1-8, wherein the polynucleotide is packaged in a vector for delivery.

10. The engineered polynucleotide of claim 9, wherein the vector is a retroviral vector.

11. A fusion protein encoded by the polynucleotide of any one of claims 1-10.

12. A host cell comprising the engineered polynucleotide of any one of claims 1-10 or the fusion protein of claim 11.

13. The host cell of claim 12, wherein the host cell is a T cell.

14. A pharmaceutical composition comprising the engineered polynucleotide of any one of claims 1-10, the fusion protein of claim 11, or the host cell of claim 12 or 13, and a pharmaceutically acceptable delivery vehicle.

15. A method of treating a cancer in a subject, the method comprising administering to the subject a therapeutically effective amount of the pharmaceutical composition of claim 14.

16. The method of claim 15, wherein the cancer is glioblastoma.

17. A method of altering a tumor microenvironment in a subject, the method comprising administering to the subject the pharmaceutical composition of claim 14 in an amount effective to alter the tumor microenvironment.

18. The method of claim 17, wherein altering the tumor microenvironment comprises reducing the amount of myeloid-derived suppressor cells in the tumor microenvironment.

19. The method of claim 17, wherein altering the tumor microenvironment comprises reducing the levels of at least one of IL10, arginase 1, and TGF-β.

20. The method of claim 17, wherein altering the tumor microenvironment comprises increasing the frequency of NK cells and B cells in the tumor microenvironment.

21. An engineered polynucleotide encoding
a chimeric antigen receptor, the chimeric antigen receptor comprising a single chain antibody to IL13Rα2 (scFv); and
a secretory interleukin 15 (IL15s).

22. The engineered polynucleotide of claim 21, wherein the polynucleotide is operably linked to an exogenous promoter sequence capable of expressing the polynucleotide in a host cell.

23. The engineered polynucleotide of any one of claims 21-22, wherein the polynucleotide is packaged in a vector for delivery.

24. The engineered polynucleotide of claim 23, wherein the vector is a retroviral vector.

25. A protein encoded by the polynucleotide of any one of claims 21-24.

26. A host cell comprising the engineered polynucleotide of any one of claims 21-24, or the protein of claim 26.

27. The host cell of claim 26, wherein the host cell is a T cell.

28. A pharmaceutical composition comprising the engineered polynucleotide of any one of claims 21-24, the protein of claim 25, or the host cell of claim 26 or 27, and a pharmaceutically acceptable delivery vehicle.

29. A method of treating a cancer in a subject, the method comprising administering to the subject a therapeutically effective amount of the pharmaceutical composition of claim 28.

30. The method of claim 29, wherein the cancer is glioblastoma.

31. A method of altering a tumor microenvironment in a subject, the method comprising administering to the subject the pharmaceutical composition of claim 28 in an amount effective to alter the tumor microenvironment.

32. The method of claim 31, wherein altering the tumor microenvironment comprises reducing the amount of myeloid-derived suppressor cells in the tumor microenvironment.

33. The method of claim 31, wherein altering the tumor microenvironment comprises reducing the levels of at least one of IL10, arginase 1, and TGF-β.

34. The method of claim 31, wherein altering the tumor microenvironment comprises increasing the frequency of NK cells and B cells in the tumor microenvironment.
